SEQUENCE LISTING (36 sequences)

SEQ ID NO: 1 - RNA, 19 nt, Artificial sequence
  Synthetic RNA oligonucleotide encoding IRF7 siRNA no. 1
ccaacagucucuacgaaga

SEQ ID NO: 2 - RNA, 19 nt, Artificial sequence
  Synthetic RNA oligonucleotide encoding IRF7 siRNA no. 2
ccagaugcguguuccugua

SEQ ID NO: 3 - RNA, 19 nt, Artificial sequence
  Synthetic RNA oligonucleotide encoding IRF7 siRNA no. 3
gagcgaagaggcuggaaga

SEQ ID NO: 4 - RNA, 19 nt, Artificial sequence
  Synthetic RNA oligonucleotide encoding IRF7 siRNA no. 4
gcccucugcuuucuaguga

SEQ ID NO: 5 - DNA, 24 bp, Artificial sequence
  Single strand DNA oligonucleotide
agcatacaggtcctggcatcttgt

SEQ ID NO: 6 - DNA, 24 bp, Artificial sequence
  Single strand DNA oligonucleotide
caaagaccacatgcttgccatcca

SEQ ID NO: 7 - DNA, 21 bp, Artificial sequence
  Single strand DNA oligonucleotide
cttcagcactttcttccgaga

SEQ ID NO: 8 - DNA, 20 bp, Artificial sequence
  Single strand DNA oligonucleotide
tgtagtgtggtgacccttgc

SEQ ID NO: 9 - DNA, 21 bp, Artificial sequence
  Single strand DNA oligonucleotide
ggtggagcactaatcaggaaa

SEQ ID NO: 10 - DNA, 20 bp, Artificial sequence
  Single strand DNA oligonucleotide
cggatatccttgagggtttg

SEQ ID NO: 11 - DNA, 23 bp, Artificial sequence
  Single strand DNA oligonucleotide
cccatgtgaaacatttcaatttc

SEQ ID NO: 12 - DNA, 18 bp, Artificial sequence
  Single strand DNA oligonucleotide
tgggcccagatcacacat

SEQ ID NO: 13 - DNA, 20 bp, Artificial sequence
  Single strand DNA oligonucleotide
ccaagaggaccaatgctcag

SEQ ID NO: 14 - DNA, 22 bp, Artificial sequence
  Single strand DNA oligonucleotide
ggcttttctcaagacttcatcc

SEQ ID NO: 15 - DNA, 23 bp, Artificial sequence
  Single strand DNA oligonucleotide
ccctcacactcagatcatcttct

SEQ ID NO: 16 - DNA, 19 bp, Artificial sequence
  Single strand DNA oligonucleotide
gctacgacgtgggctacag

SEQ ID NO: 17 - DNA, 20 bp, Artificial sequence
  Single strand DNA oligonucleotide
gactccagccacactccaac

SEQ ID NO: 18 - DNA, 18 bp, Artificial sequence
  Single strand DNA oligonucleotide
tgacagcgcagctcattg

SEQ ID NO: 19 - DNA, 26 bp, Artificial sequence
  Single strand DNA oligonucleotide
aaaatcatccaaaagatactgaacaa

SEQ ID NO: 20 - DNA, 20 bp, Artificial sequence
  Single strand DNA oligonucleotide
ctttggttcttccgttgagg

SEQ ID NO: 21 - DNA, 21 bp, Artificial sequence
  Single strand DNA oligonucleotide
tttgaattccctgggtgagaa

SEQ ID NO: 22 - DNA, 20 bp, Artificial sequence
  Single strand DNA oligonucleotide
ggagaaatcgatgacagcgc

SEQ ID NO: 23 - DNA, 18 bp, Artificial sequence
  Single strand DNA oligonucleotide
ctttgccacggacgagac

SEQ ID NO: 24 - DNA, 22 bp, Artificial sequence
  Single strand DNA oligonucleotide
tcattgtactctgagggctgac

SEQ ID NO: 25 - DNA, 20 bp, Artificial sequence
  Single strand DNA oligonucleotide
caaatcgtggtcatcgtggt

SEQ ID NO: 26 - DNA, 20 bp, Artificial sequence
  Single strand DNA oligonucleotide
ttccacctgacaccgtactc

SEQ ID NO: 27 - DNA, 23 bp, Artificial sequence
  Single strand DNA oligonucleotide
tgcaagagacttccatccagttg

SEQ ID NO: 28 - DNA, 24 bp, Artificial sequence
  Single strand DNA oligonucleotide
taagcctccgacttgtcaagtggt

SEQ ID NO: 29 - DNA, 24 bp, Artificial sequence
  Single strand DNA oligonucleotide
acctgtcctgtgtaatgaaagacg

SEQ ID NO: 30 - DNA, 20 bp, Artificial sequence
  Single strand DNA oligonucleotide
tgggtattgcttgggatcca

SEQ ID NO: 31 - DNA, 21 bp, Artificial sequence
  Single strand DNA oligonucleotide
aactgcatccatatcgatgac

SEQ ID NO: 32 - DNA, 19 bp, Artificial sequence
  Single strand DNA oligonucleotide
gtggcaatgatctcaacac

SEQ ID NO: 33 - DNA, 21 bp, Artificial sequence
  Single strand DNA oligonucleotide
cgagaggcagagatttatcag

SEQ ID NO: 34 - DNA, 22 bp, Artificial sequence
  Single strand DNA oligonucleotide
atgtcccattgtctttgttgtc

SEQ ID NO: 35 - DNA, 21 bp, Artificial sequence
  Single strand DNA oligonucleotide
tttcggaagaatacaccacca

SEQ ID NO: 36 - DNA, 22 bp, Artificial sequence
  Single strand DNA oligonucleotide
atgatgacagctatggcaatcc

(19) United States
(12) Patent Application Publication
(10) Pub. No.: US 2017/0348393 A1
(43) Pub. Date: Dec. 7, 2017

(54) TREATMENT OF CNS INFLAMMATORY DISORDERS

(71) Applicant: Yeda Research And Development Co., Ltd., Rohovot (IL)

(72) Inventors: Michal SCHWARTZ-EISENBACH, Rohovot (IL); Ido AMIT, Rohovot (IL); Merav COHEN, Rohovot (IL); Orit MATCOVICH, Rohovot (IL)

(21) Appl. No.: 15/524,634

(22) PCT Filed: Nov. 5, 2015
(86) PCT No.: PCT/IL2015/051072

§ 371 Date: May 5, 2017

Related U.S. Application Data
(60) Provisional application No. 62/075,923, filed on Nov. 6, 2014.

(51) Int. Cl. A61K 38/21

(52) U.S. Cl. CPC A61K 38/215

(57) ABSTRACT

A method of upregulating an anti-inflammatory response in a central nervous system (CNS) of a subject in need thereof is disclosed. The method comprising locally administering to the CNS of the subject a therapeutically effective amount of IFN-beta, thereby upregulating the anti-inflammatory response in the CNS of the subject. Methods of treating an inflammation in a CNS or treating a disease, disorder, condition or injury of a CNS of a subject are also disclosed.

### FIELD AND BACKGROUND OF THE INVENTION

The present invention, in some embodiments thereof, relates to the treatment of central nervous system inflammatory disorders and, more particularly, but not exclusively, to the use of IFN-β for treating diseases, disorders, conditions or injuries of the CNS.

Resident microglia are the major specialized innate immune cells of the central nervous system (CNS). Following CNS injury, both brain resident myeloid cells (microglia), and infiltrating monocyte-derived macrophages (mo-MΦ), are present at the site of injury. These two cell populations differ in their function and origin. While the microglia are derived from primitive yolk-sac myeloid progenitors that arise before day 8 of embryogenesis, the mo-MΦ are derived primarily from the bone marrow. In addition, differentiation of each of these cell types requires an overlapping, though non-identical set of transcription factors (TF).

In general, the appropriate differentiation of macrophages to a classical inflammatory activated (M1) state or alternative suppressive (M2) state is critical for tissue homeostasis and immune clearance. During the process of wound healing or pathogen removal, monocytes infiltrate the damaged tissue, leading to a transient inflammatory response (M1) that is resolved either via local conversion to M2-like macrophages, or through additional recruitment of anti-inflammatory cells.

Following acute injury, there is an immediate and crucial phase of microglial activation in the CNS, however, these cells fail to acquire an inflammation-resolving phenotype (M2-like phenotype) in a timely manner, often resulting in self-perpetuating local inflammation and tissue destruction beyond the primary insult. Under such injurious conditions, recruitment of mo-MΦ or bone-marrow derived monocytes to the lesion site was found to have a pivotal role in the repair process by resolving the microglial-induced inflammation. However, why microglia, unlike mo-MΦ, fail to acquire an anti-inflammatory phenotype under such pathological conditions remains an enigma.

It is conceivable that the limited ability of resident microglia to acquire an M2-like phenotype is either an inherent aspect of the microglial differentiation program or an outcome of the unique CNS microenvironment to which they are chronically exposed, as these cells have limited capacity for self-renewal. In this context, it is important to note that the CNS microenvironment is characterized by enrichment of anti-inflammatory factors such as IL-13, IL-4, and members of the transforming growth factor β (TGF-β) family, recently shown to be manifested as a signature of adult microglial markers during homeostasis. Whether and how the chronic exposure to TGF-β imprints microglial activity under pathological conditions has not been investigated. The TGF-β subfamily includes TGF-β1, -2, and -3, whose expression is abundant in the CNS. TGF-β1 expression by astrocytes, microglia and neurons is up-regulated following CNS insult, and is also upregulated during aging. Moreover, TGF-β1 is involved in mitigating inflammation, promoting resolution [Huynh et al., The Journal of clinical investigation (2002) 109: 41-50], and is highly expressed relative to the other isoforms in the spinal cord following spinal cord injury (SCI) [Shechter R. et al., Immunity (2013) 38: 555-569].

European Patent Application no. EP 1716235 (to Bogdahn U. et al.) provides antisense oligonucleotides inhibiting the expression of TGF-receptor for the prevention or treatment of CNS disorders (e.g. traumatic brain and spinal cord injuries).

U.S. Patent Application no. 20080031911 (to He Z. et al.) provides methods of promoting regeneration of lesioned CNS axon of a mature neuron, determined to be subject to regeneration inhibition by Smad2/3 mediated TGF-beta signaling, by contacting the neuron with an inhibitor of Smad2/3 signaling sufficient to promote regeneration of the axon. According to U.S. 20080031911, a preferred inhibitor is an activin inhibitor, an activin receptor-like kinase (ALK) inhibitor or a Smad2/3 inhibitor.

U.S. Patent Application no. 20020169102 (to Frey et al.) provides a method of regulating the development of a donor cell in the central nervous system of a mammal. The method comprises administering a composition comprising a therapeutically effective amount of at least one regulatory agent (e.g. a growth factor such NGF or IGF-I, or a cytokine such as IFN-β or IFN-γ) to a tissue of the mammal innervated by the trigeminal nerve and/or the olfactory nerve. The method may be used for the treatment and/or prevention of CNS disorders, such as, brain and spinal cord injuries.

### SUMMARY OF THE INVENTION

According to an aspect of some embodiments of the present invention there is provided a method of upregulating an anti-inflammatory response in a central nervous system (CNS) of a subject in need thereof, the method comprising locally administering to the CNS of the subject a therapeutically effective amount of IFN-β, thereby upregulating the anti-inflammatory response in the CNS of the subject.

According to an aspect of some embodiments of the present invention there is provided a method of treating an inflammation in a CNS of a subject in need thereof, the method comprising locally administering to the CNS of the subject a therapeutically effective amount of IFN-β, thereby treating the inflammation in the CNS of the subject.

According to an aspect of some embodiments of the present invention there is provided a method of treating a disease, disorder, condition or injury of a CNS in a subject in need thereof, the method comprising locally administering to the CNS of the subject a therapeutically effective amount of IFN-β, thereby treating the disease, disorder, condition or injury of the CNS in the subject.

According to an aspect of some embodiments of the present invention there is provided a use of a therapeutically effective amount of IFN-β in the manufacture of a medicament for treating an inflammation in a CNS of a subject in need thereof, wherein the medicament is formulated for local administration to the CNS.

According to an aspect of some embodiments of the present invention there is provided a use of a therapeutically effective amount of IFN-β in the manufacture of a medicament for treating a disease, disorder, condition or injury of a CNS in a subject in need thereof, wherein the medicament is formulated for local administration to the CNS. According to some embodiments of the invention, the therapeutically effective amount upregulates the activity or expression of IRF7.

According to some embodiments of the invention, the therapeutically effective amount downregulates the expression of at least one pro-inflammatory associated gene.

According to some embodiments of the invention, the pro-inflammatory associated gene is selected from the group consisting of iNos, Tnfα, Il-1β, Il-6, Cxcl1, Cxcl2 and Cxcl10.

According to some embodiments of the invention, the therapeutically effective amount upregulates the expression of at least one anti-inflammatory associated gene.

According to some embodiments of the invention, the anti-inflammatory associated gene is selected from the group consisting of IL-10, MMR (CD206), CD36, DECTIN-1, IL-4 and IL-13.

According to some embodiments of the invention, the therapeutically effective amount induces a M1-to-M2 phenotype conversion of a myeloid cell.

According to some embodiments of the invention, the myeloid cell comprise a microglia cell.

According to some embodiments of the invention, the locally administering is to a parenchymal tissue of the CNS.

According to some embodiments of the invention, the locally administering is effected by a route selected from the group consisting of intracranial (IC), intracerebroventricular (ICV), intrathecal and intraparenchymal CSF administration.

According to some embodiments of the invention, the subject is a human subject.

According to some embodiments of the invention, the subject has a neurodegenerative disorder or a neuroinflammatory disorder.

According to some embodiments of the invention, the subject has a disease, disorder, condition or injury of a CNS.

According to some embodiments of the invention, the disease, disorder, condition or injury of the CNS is selected from the group consisting of spinal cord injury, closed head injury, blunt trauma, penetrating trauma, hemorrhagic stroke, ischemic stroke, cerebral ischemia, spinal ischemia, optic nerve injury, myocardial infarction.

According to some embodiments of the invention, the IFN-β is soluble.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-T illustrate that M2-like phenotype acquired by NB-Mg under ex vivo conditions can be inhibited by TGF-β1 preconditioning. (FIG. 1A) Total RNA was harvested from NB-Mg following various treatments: Cells were left untreated (marked as M), or stimulated with LPS (100 ng/ml) for 4 hours [marked as LPS(4 h), grey], or stimulated with LPS for 20 hours [marked as LPS(20 h)], or stimulated with LPS for 20 hours, washed with growth medium, and re-challenged with LPS for an additional 4 hours [marked as LPS(20 h/4 h), orange], or preconditioned for 20 hours with different anti-inflammatory cytokines, and then stimulated with LPS for 20 hours, washed, and re-challenged with LPS for an additional 4 hours [marked as Pre(20 h)/LPS(20 h/4 h), green]. (FIGS. 1B-J) NB-Mg were stimulated as described in FIG. 1A, and RNA was analyzed by RT-qPCR for the expression of representative pro- and anti-inflammatory genes. (FIGS. 1K-O) NB-Mg were stimulated as described in FIG. 1A, and preconditioned with TGF-β1 (100 ng/ml), IL-4 (10 ng/ml), IL-13 (10 ng/ml), or TGF-β2 (100 ng/ml). RNA was analyzed by RT-qPCR for the expression of representative pro- and anti-inflammatory genes. Results are normalized to the expression of the housekeeping gene, peptidylprolyl isomerase A (PPIA), and expressed as fold increase relative to the mRNA levels of untreated cells (M). Asterisks are relative to the [LPS(20 h/4 h)] sample, unless indicated otherwise. (FIGS. 1P-R) Mice were injured, and parenchymal segments of 0.5 mm from each side of the spinal cord lesion site were excised on the indicated days following SCI. Cells purified from the injured spinal cord parenchyma (n=6) were incubated in growth medium (see general materials and experimental procedures section herein below) for 3 hours, and then washed and stained for the intracellular cytokine IL-10. (FIGS. 1S-T) Flow cytometry quantification of percentage and number of mo-MΦ and resident microglia that expressed IL-10 at day 7 following SCI. Data were normalized to 10,000 cells. Student's t test for percent of IL-10+ cells, p=0.034; number of IL-10+ cells, p=0.047. Samples were prepared in triplicates, and results are representative of at least three different experiments. *p<0.05, **p<0.01, ***p<0.005. Data are represented as mean±SEM.

FIGS. 2A-K illustrate that a long exposure to TGF-β1 activates a robust gene expression program in myeloid cells. Total gene expression during a time course of 0, 1, 3, 6, 10, and 20 hours following TGF-β1 (100 ng/ml) treatment in BM-MΦ and NB-Mg, analyzed by RNA-seq. (FIG. 2A) mRNA expression profile of genes, whose expression level was elevated or reduced 2 fold in at least one of the time points in either TGF-β1 stimulated BM-MΦ or NB-Mg. Genes were clustered according to k-mean of 20 (red, high relative expression; white, mean expression; blue, low relative expression). (FIGS. 2B-C) Distributions of the number of genes expressed in BM-MΦ (blue) and NB-Mg (red) that were increased (FIG. 2C) or decreased (FIG. 2B) along the time course (Kolmogorov-Smirnov test, p-value<10-5). (FIGS. 2D-E) Expression levels of Tgfbr1 in BM-MΦ (blue) and NB-Mg (red) along the time-course of TGF-β1 treatment was analyzed by RNA-seq. (FIG. 2F) Peripheral CX3CR1lowLy6C+ monocytes and resident microglia were sorted from non-injured CX3CR1GFP/+ mice, and the expression of Tgfbr1 and Tgfbr2 was analyzed using RT-qPCR. RT-qPCR results are normalized to the expression of PPIA. (FIG. 2G) eGFP>WT chimeric mice were injured and parenchymal segments of 0.5 mm from each side of the spinal cord lesion site were excised on different days following spinal cord injury (SCI). GFP+mo-MΦ and GFP-resident microglia were sorted by FACS, and collected directly into lysis buffer. RNA was harvested and gene expression profile was analyzed by RNA-seq. (FIGS. 2H-I) Venn diagrams of genes that were: up-regulated (FIG. 2H) or down-regulated (FIG. 2I) in BM-MΦ at least 2 fold by exposure to TGF-β1 ex vivo (red), and genes from the in vivo kinetic studies whose expression was significantly different (p-value<0.05) between microglia and mo-MΦ, selecting those that were expressed to a higher (FIG. 2H) or lower (FIG. 2I) extent in microglia compared to mo-MΦ along the kinetics following SCI (blue), respectively; Hypergeometric test for the intersection of up-regulated genes, p-value<10-5; Hypergeometric test for the intersection of down-regulated genes, p-value=3×10⁻³; n=12 for all kinetic following SCI. (FIG. 2J) Expression profile of genes that were down-regulated at least 2 fold by BM-MΦ following exposure to TGF-β1 ex vivo, and were highly expressed by mo-MΦ compared to microglia (2 fold), and (FIG. 2K) expression profile of genes that were up-regulated by BM-MΦ at least 2 fold following exposure to TGF-β1 ex vivo, and were highly expressed by microglia (2 fold) compared to mo-MΦ at days 3 and 7 following SCI, divided into functional groups; Red, high relative expression; white, mean expression; blue, low relative expression. Data represent the average expression in two independent experiments; each experiment was performed in duplicate.

FIGS. 3A-M illustrate that pro- to anti-inflammatory phenotype switch is regulated by IRF7, which is suppressed by TGF-β1. (FIG. 3A) Left panel—RNA-seq expression profile of transcription factor genes, whose expression level was induced or reduced by a factor of 2 on at least one of the time points in either BM-MΦs or NB-Mg stimulated with TGF-β1 (100 ng/ml) along a time course of 0, 1, 3, 6, 10, and 20 hours. Genes were clustered according to their time to peak. Cluster numbers (I-XII) were noted on the right and cluster size was indicated in parentheses; representative member genes were identified on the left; red, high relative expression; white, mean expression; blue, low relative expression. Right panel—mean relative expression profiles for each cluster were calculated at each time point of the kinetics. (FIGS. 3B-E) Gene expression profile of Irf7 and Pparγ in BM-MTs (blue) and NB-Mg (red) was analyzed from RNA-seq data. Data represent the average expression of two independent experiments; each experiment was performed in duplicate. (FIG. 3F) RT-qPCR analysis of Irf7 expression in BM-MΦ (blue) and NB-Mg (red) following 20 hour treatment with TGF-β1 or with growth medium. (FIG. 3G) BM-MTs (blue) and NB-Mg (red) were stimulated with LPS (100 ng/ml) for 20 hours, washed, and re-challenged with LPS for 4 hours. RNA was harvested along the time course of 0, 1, 2, 4, 10, 20 hours of the first LPS stimulation and 0, 1, 2, 4 hours of the second LPS stimulation, and gene expression profile of Irf7 was analyzed by RNA-seq. (FIGS. 3H-I) RT-qPCR analysis of Irf7 expression in BM-MΦ (FIG. 3I) and NB-Mg (FIG. 3H) after long stimulation with LPS followed by LPS re-challenge [marked as LPS(20 h/4 h)], with (green) or without (orange) 20 hours preconditioning with TGF-β1. (FIG. 3J) BM-MΦ were transfected with siRNA directed against Irf7 or scrambled, and treated for 20 hours with LPS, washed, and re-challenged with LPS [marked as LPS(20 h/4 h)]; RNA was harvested and analyzed using RT-qPCR. Results are shown as change in gene expression between siIrf7-treated cells and controls. Asterisks indicate significance of the differences between siIrf7 treatment and scrambled controls for each gene. (FIG. 3K) Peripheral CX3CR1lowLy6C+ monocytes and resident microglia were sorted by FACS from non-injured CX3CR1GFP/+ mice (n=3), and the expression of Irf7 was analyzed using RT-qPCR. (FIGS. 3L-M) GFP+mo-MΦ and GFP-resident microglia were sorted from GFP>WT chimeric mice by FACS. RNA was harvested and gene expression level was analyzed by RT-qPCR. (FIG. 3L) Irf7 expression in mo-MΦ (blue) and microglia (red) was analyzed in non-injured mice, and at 16 hours, day 1, day 3, day 7 and day 14 following SCI (n=2 at each time point). (FIG. 3M) Correlation between Irf7 and 1/Tgfβr1 expression level by microglia in homeostasis and following SCI (r2=0.84). Data represent results of one, out of two independent experiments. *p<0.05, **p<0.01, ***p<0.005; FIGS. 3F, 3H-K—results are normalized to the expression of PPIA; Data are represented as mean±SEM.

FIGS. 4A-J illustrate that IRF7 regulates the M1-to-M2 phenotype switch by down-regulation of pro-inflammatory gene expression. (FIGS. 4A-D) ChIP-Seq of Irf7 was performed on GM-CSF induced bone marrow cells (stimulated for 2 hours with LPS or untreated controls). (FIG. 4A) Venn diagram of genes whose expression in vivo by mo-MΦ was either elevated (M2-related genes, orange) or decreased (M1-related genes, green) at day 7 relative to the first 3 days following SCI (p-value<0.05), and genes whose promoters were bound by Irf7 (blue), (Hypergeometric test: green-blue intersection, p-value<10-5; orange-blue intersection p-value=0.4). (FIG. 4B) Pie chart dividing the M1-related genes, whose promoters were bound by Irf7 (green-blue intersection) to functional groups using the PANTHER database of gene ontology (see materials and experimental procedures section herein below). (FIGS. 4C-D) Expression profile of transcription regulation genes (FIG. 4C), and immune response genes (FIG. 4D) out of M1-related genes, which were bound by Irf7 and down-regulated in mo-MΦ along the time course following SCI. (FIGS. 4E-J) ChIP-Seq signal intensity of selected pro-inflammatory genes was represented by sequencing (FIGS. 4E, 4G and 4I), and their in vivo expression in microglia (red) and mo-MΦ (blue) at days 3 and 7 following SCI is represented in FIGS. 4F, 4H and 4J.

FIGS. 5A-M illustrate that IFN-β1 can overcome the inability of microglial phenotype switch, through elevation of IRF7. (FIGS. 5A-F) Total RNA was harvested from NB-Mg that were stimulated with LPS for 20 hours, washed, and re-challenged with LPS for 4 hours [marked as LPS(20 h/4 h), empty orange bars], or preconditioned with TGF-β1 for 20 hours prior to LPS(20 h/4 h) stimulation [marked as TGβ1(20 h)/LPS(20 hr/4 h), empty green bars], or preconditioned with TGF-β1 for 20 hours, and also stimulated with IFN-β1 (1000 U/ml) starting from 1 hour prior to the 4 hours re-challenge with LPS [marked as TGFβ1(20 h)/LPS(20 h)/IFNβ/LPS(4 h), filled green bars]. RNA was analyzed by RT-qPCR for the expression of (FIG. 5B) Irf7, and (FIGS. 5C-F) representative pro- and anti-inflammatory genes. Asterisks indicate significance relative to the [TGFβ1(20 h)/LPS(20 hr/4 h)] samples. Samples were prepared in triplicates, and results are representative of at least two different experiments. (FIGS. 5G-I) GFP>WT chimeric mice were injured and intra-parenchymal injected 24 hours later with IFN-β1 (800 U/25 gr) or PBS 24 hours following SCI. Parenchymal segments of the spinal cord lesion site (0.3 mm on both sides of the lesion) were excised 48 hours and 72 hours following SCI. Il-1β and Tnfα expression in microglia was analyzed in non-injured mice, and at 1 hour, 16 hours, day 1 and day 3 following SCI by RNA-seq (n=2 at each time point). (FIGS. 5J-M) GFP-activated microglia were sorted by FACS at 48 hours (n=4) and 72 hours (n=10; pool of three different experiments) following SCI, RNA was extracted and gene expression levels of Il-1β (p-value (48 h)=0.013; p-value (72 h)=0.026) and Tnfα (p-value (48 h)=0.019; p-value (72 h)=0.034) were analyzed by RT-qPCR. *p<0.05, **p<0.01, ***p<0.005. FIGS. 5B, 5C-F, 5J-M—results are normalized to the expression of PPIA. Data are represented as mean±SEM.

FIGS. 5N-R illustrate gene expression profile of sorted activated microglia and mo-MΦ following SCI. (FIGS. 5N-Q) exhibit flow cytometry plots characterizing the gates of sorted mo-MΦ, GFP^{+Ly}6 G⁻CD11b⁺CD45.2⁺, and activated microglia, GFP⁻CD11b⁺CD45.2⁺. (FIG. 5R) GFP>WT chimeric mice were injured and intra-parenchymal injected with IFN-β1 (800 U/25 gr) or PBS 24 hours following SCI. Parenchymal segments of 0.3 mm from each side of the spinal cord lesion site were excised 36 hours following SCI. GFP⁻ activated microglia were sorted by FACS, RNA was extracted and gene expression level of Irf7 was analyzed by RT-qPCR (n=6; p-value=0.005). ***p<0.005. Results are normalized to the expression of PPIA. Data are represented as mean±SEM.

FIGS. 6A-C are a schematic model depicting the molecular mechanism explaining the perturbed M1-to-M2 switch by microglia. (FIG. 6A) During homeostasis, the CNS microenvironment is enriched with the anti-inflammatory cytokines TGF-β1 and TGF-β2, and adult microglia express their relevant receptors. As a result of chronic exposure to a TGF-β1 enriched microenvironment, the mRNA levels of Irf7 are down-regulated in microglia, and the anti-viral program is shut-off; consequently, the transcription of IRF7-induced genes is suppressed. (FIG. 6B) A CNS insult results in the activation of resident microglia and a robust M1 response, characterized by the induction of the inflammatory program (NF-kB) and the transcription of pro-inflammatory cytokines such as Tnfα, Il1β, Cxcl1, Cxcl2 and the down-regulation of Il10 expression. The low expression levels of Irf7, resulting from long microglial exposure to TGF-β1, prevents the switch to M2 anti-inflammatory phenotype, and leads to a vicious cycle of the M1 response in adult microglia. (FIG. 6C) Treatment of the TGF-β1-imprinted microglia, under inflammatory conditions, with IFN-β1, induces Irf7 expression and consequently, the expression of IRF7-associated genes. IRF7 induction rescues the switch from M1 to M2 phenotype, possibly through inhibition of the NFkB pathway. The up-regulation of IRF7 results in direct suppression of pro-inflammatory gene expression (e.g. Tnfα, Il1β, Cxcl1 and Cxcl2), and in indirect induction of Il10 transcripts. Dotted lines (small black shape) denote pathways that are not fully characterized in this study; dotted lines (long gray shape) denote the suggested pathway.

FIG. 7 is a schematic model depicting a synopsis scheme. Chronic exposure to the abundant CNS cytokine, TGFβ1, impairs the ability of myeloid-cells, specifically microglia, to acquire an inflammation-resolving, anti-inflammatory (M2), phenotype under pathological conditions. The transcription factor IRF7 is a key regulator of the M1-to-M2 phenotype switch and is down-regulated by the TGFβ1 signaling pathway. Induction of IRF7 expression by IFN-β1 under pathological conditions reduces microglial pro-inflammatory response following injury and enables the M2 phenotype switch.

FIGS. 8A-I illustrate that M2-like phenotype acquired by NB-Mg and BM-MΦ under ex vivo conditions can be prevented by TGF-β1 preconditioning. (FIGS. 8A-C) Total RNA was harvested from BM-MΦ following different treatments: Cells were stimulated with LPS (100 ng/ml) for 4 hours [marked as LPS(4 h)], or stimulated with LPS for 20 hours, washed with growth medium, and re-challenged with LPS for additional 4 hours, with [marked as LPS(20 h/4 h), green] or without [marked as LPS(20 h/4 h), red] preconditioning with TGF-β1 (100 ng/ml) for 20 hours. RNA was analyzed by RT-qPCR for the expression of representative pro-inflammatory genes. (FIGS. 8D-I) NB-Mg and BM-MΦ were preconditioned with TGF-β1 and stimulated as described in FIGS. 8A-C. RNA was analyzed by RT-qPCR for the expression of representative genes whose expression by tolerant NB-Mg and BM-MΦ did not change following TGF-β1 preconditioning. Results are normalized to the expression of PPIA, and expressed as fold increase relative to mRNA levels of untreated cells. Samples were prepared in triplicates or quadruplicates, and results are representative of at least three different experiments. *p<0.05, **p<0.01, ***p<0.005, asterisks are relative to [LPS(20 h/4 h)] sample, unless noted differently; Data are represented as mean±SEM.

FIGS. 9A-I illustrate that long exposure to TGF-β1 activates a robust gene expression program in myeloid cells, with implications to CNS pathological conditions. RNA-seq analysis of mRNA expression profile of genes, whose expression level was induced or reduced by a factor of 2 on at least one of the time points in either BM-MΦ or NB-Mg stimulated with TGF-β1 (100 ng/ml) along a time course of 0, 1, 3, 6, 10, and 20 hours. (FIG. 9A) mRNA expression profile of genes that are related to matrix metalloproteinase activity; representative genes are noted on the left. (FIGS. 9B-D) (FIG. 9B) mRNA expression profile of genes that are related to phagocytosis, pinocytosis, endocytosis or exocytosis processes. Genes were clustered according to their time to peak. Cluster numbers (I-XII) are noted on the right; representative genes are noted on left. (FIG. 9C) The mean relative expression profiles for each cluster were calculated at each time point along the kinetics. (FIG. 9D) RT-qPCR analysis of Anxal expression in BM-MΦ (blue) and NB-Mg (red) following a 20 hour treatment with TGF-β1 or with growth medium. (FIGS. 9E-I) (FIG. 9E) mRNA expression profile of genes that are related to immune response processes and regulation. Genes were clustered according to their time to peak. Cluster numbers (I-XII) are noted on the right; representative genes are identified on the left. (FIG. 9F) The mean relative expression profiles for each cluster were calculated at each time point along the kinetics. (FIGS. 9G-I) RT-qPCR analysis of representative genes in BM-MΦ (blue) and NB-Mg (red) (Lgals3, Ccl9, Pmepa1) following a 20 hour treatment with TGF-β1 or with growth medium. (FIGS. 9B-I) The size of each cluster is indicated in parentheses; (FIGS. 9A, 9B and 9C), red, high relative expression; white, mean expression; blue, low relative expression. (FIGS. 9D, 9G-I) The results are pooled from three independent experiments, normalized to the expression of PPIA, and expressed as fold increase relative to mRNA levels of untreated cells. Samples were prepared in triplicates or quadruplicates. *p<0.05, **p<0.01, ***p<0.005; Data are represented as mean±SEM.

### DESCRIPTION OF SPECIFIC EMBODIMENTS OF THE INVENTION

The present invention, in some embodiments thereof, relates to the treatment of central nervous system inflammatory disorders and, more particularly, but not exclusively, to the use of IFN-β for treating diseases, disorders, conditions or injuries of the CNS.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Resident microglia are the exclusive innate immune cells of the central nervous system (CNS), and maintain normal CNS function during homeostasis. However, under severe acute- or chronic-activation, activated microglia may become neurotoxic over time, as they fail to undergo self-resolution of their inflammatory phenotype. Under such conditions, the inflammation-resolving function in the CNS is dependent on peripheral assistance from infiltrating monocyte-derived macrophages (mo-MΦ).

Transforming Growth Factor-β1 (TGF-β1) is among the molecules that constitutively support adult CNS maintenance by contributing to the life-long anti-inflammatory milieu. However, in contrast to the anti-inflammatory effect of short exposure to TGFβ1, the present invention illustrates that continuous exposure to TGF-β1 has significant drawbacks under severe and potentially chronic inflammatory conditions.

Thus, while reducing the present invention to practice, the present inventors have uncovered that long exposure to TGF-β1 impaired the ability of myeloid-cells to acquire a resolving anti-inflammatory phenotype (see Example 1 of the Examples section which follows). Using genome-wide expression analysis and chromatin immunoprecipitation followed by next generation sequencing, the present inventors showed that the capacity to undergo pro- to anti-inflammatory (M1-to-M2) phenotype switch is controlled by the transcription factor Interferon regulatory factor-7 (IRF7) that is down-regulated by the TGF-β1 pathway (see Example 3 of the Examples section which follows). RNAi-mediated perturbation of Irf7 inhibited the M1-to-M2 switch (see Example 3 of the Examples section which follows), while IFN-β1 (an IRF7 pathway activator) restored it (see Example 4 of the Examples section which follows). Moreover, in vivo induction of Irf7 expression in microglia, following spinal cord injury, reduced their pro-inflammatory activity (see Example 4 of the Examples section which follows).

Taken together, these results exemplify that the fate of CNS resident myeloid-derived cells (e.g. under pathological conditions) can be shifted from a pro-inflammatory phenotype (M1) to an anti-inflammatory phenotype (M2) by induction of IRF7. Such a therapeutic modality may be used for the treatment of pathologies associated with CNS inflammation and damage, such as CNS injury.

Thus, according to one aspect of the present invention there is provided a method of upregulating an anti-inflammatory response in a central nervous system (CNS) of a subject in need thereof, the method comprising locally administering to the CNS of the subject a therapeutically effective amount of IFN-β, thereby upregulating the anti-inflammatory response in the CNS of the subject.

According to another aspect of the present invention there is provided a method of treating an inflammation in a CNS of a subject in need thereof, the method comprising locally administering to the CNS of the subject a therapeutically effective amount of IFN-β, thereby treating the inflammation in the CNS of the subject.

According to another aspect of the present invention there is provided a method of treating a disease, disorder, condition or injury of a CNS in a subject in need thereof, the method comprising locally administering to the CNS of the subject a therapeutically effective amount of IFN-β, thereby treating the disease, disorder, condition or injury of the CNS in the subject.

According to another aspect of the present invention there is provided a use of a therapeutically effective amount of IFN-β in the manufacture of a medicament for treating an inflammation in a CNS of a subject in need thereof, wherein the medicament is formulated for local administration to the CNS.

According to another aspect of the present invention there is provided a use of a therapeutically effective amount of IFN-β in the manufacture of a medicament for treating a disease, disorder, condition or injury of a CNS in a subject in need thereof, wherein the medicament is formulated for local administration to the CNS.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology (further details are provided below).

The terms “subject” or “subject in need thereof” as used herein include mammals, preferably human beings at any age (male or female) which suffer from the pathology or who are at risk to develop the pathology (e.g. inflammation in the CNS).

As used herein, “central nervous system” or “CNS” refers to the brain and spinal cord.

An “anti-inflammatory response” according to the present invention relates to putting off, delaying, slowing, inhibiting, stopping, reducing or ameliorating anyone of the events that form the complex biological response associated with an inflammation of a central nervous system in an individual (as described below).

According to one embodiment, the anti-inflammatory response in the CNS involves the production of anti-inflammatory factors, such as but not limited to, TGF-β1, TGF-β2, IL-4, IL-10 and/or IL-13 from cells of the CNS (e.g. astrocytes). The production of anti-inflammatory factors typically results in cessation of pro-inflammatory signaling activation and consequently in microglial resting state (during homeostasis).

The terms “inflammatory response” and “inflammation” as used herein refer to the general terms for local accumulation of fluids, plasma proteins, and white blood cells (e.g. in the CNS) initiated by physical injury, trauma, infection, stress or a local immune response (e.g. in the CNS) Inflammation is an aspect of many diseases and disorders of the CNS, including but not limited to, physical injuries or traumas, diseases related to immune disorders, pathogens (e.g. viral and bacterial infections), damaged cells, or irritants, and includes secretion of cytokines and more particularly of pro-inflammatory cytokines, i.e. cytokines which are produced predominantly by activated immune cells (e.g. microglia) but also by other cells in the CNS (e.g. astrocytes, endothelial cells). Exemplary pro-inflammatory cytokines include, but are not limited to, IL-1β, IL-6, CXCL1, CXCL2 and TNF-α. Such pro-inflammatory cytokines are generally involved in the amplification of the inflammatory reaction, such as in activation of endothelial cells, platelet deposition, and tissue edema (e.g. in acute inflammation), or in sustained activation of microglia cells and recruitment of other immune cells into the brain (e.g. in chronic inflammation).

Inflammation according to the present teachings may be associated with acute (short term) inflammatory diseases or disorders or chronic (long term) inflammatory diseases or disorders. Acute inflammation indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). Chronic inflammation indicates a condition characterized by concurrent active inflammation, tissue destruction, and attempts at repair. Chronic inflammation is usually not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis.

Inflammation to the CNS can be triggered by injury, for example injury to skull or nerves Inflammation can be triggered as part of an immune response, e.g., pathologic autoimmune response involving the CNS Inflammation can also be triggered by infection, where pathogen recognition and tissue damage can initiate an inflammatory response at the site of infection (in the CNS).

According to one embodiment, the inflammation can be a sterile inflammation (i.e., as a result of an injury, trauma or stroke) or a pathogenic inflammation (i.e., caused by a pathogen such as a bacteria, virus or fungus), as discussed in detail below.

According to one embodiment, the inflammation is associated with an injury to the CNS.

Exemplary injuries to the CNS include, but are not limited to, spinal cord injury (SCI) e.g. chronic spinal cord injury, such as, but not limited to, those caused from physical trauma such as vehicle crashes, bullet wounds, falls, or sports injuries, or from diseases such as transverse myelitis, polio, spina bifida or Friedreich's ataxia; stroke, chronic deficits after stroke, hemorrhagic stroke, ischemic stroke; cerebral ischemia, cerebral infarction; chronic progressive multiple sclerosis; closed head injury; traumatic brain injury (TBI), e.g. blunt trauma, penetrating trauma, such as that caused by falls, vehicle crashes, sports injuries, shock waves (e.g. from a battlefield explosion), bullet wounds or other brain-penetrating injuries; optic nerve injury; myocardial infarction; organophosphate poisoning; injury caused by surgery (e.g. tumor excision), cancer-related brain injury, cancer-related spinal cord injury.

According to one embodiment, the inflammation is associated with an inflammatory disease.

Exemplary inflammatory diseases in the central nervous system include, but are not limited to, meningitis, meningoencephalitis, encephalitis, and encephalopathy; peripheral demyelinating neuropathies such as Guillain-Barre syndrome and chronic inflammatory demyelinating polyneuropathy; and acute central nervous system autoimmune diseases, such as neurosarcoidosis. Meningitis, meningoencephalitis, encephalitis and encephalopathy may occur due to various causes such as pathogen infection, infiltration of cancer into the central nervous system, autoimmunity and metabolic disorders (e.g. as a result of viral, bacterial, tuberculous, fungal, carcinomatous, autoimmune or metabolic causes).

Inflammation can occur at any stage of the disease (e.g. at an early stage after disease onset, e.g. within several hours, or even several days, weeks or months after disease onset).

According to a specific embodiment, the inflammation or CNS disorder is not due to cell therapy.

The term “upregulating” refers to increasing the anti-inflammatory response. According to one embodiment, the anti-inflammatory response is increased by about 10%, 20%, 30%, 40%, 50%, 60%, 70%, 80%, 90% or 100% as compared to an anti-inflammatory response in a tissue not subjected to a treatment (e.g., with IFN-β) according to some embodiments of the invention.

According to one embodiment, upregulating an anti-inflammatory response or treating an inflammation in a CNS of a subject is affected by a local administration of a therapeutically effective amount of IFN-β.

As used herein, the term “IFN-β” refers to the cytokine interferon-beta. According to a specific embodiment the IFN-β is IFN-β1. According to a specific embodiment the IFN-β1 is IFN-β1a or IFN-β1b. According to a specific embodiment, the human form of IFN-β is provided in the following: for the protein, accession number in the NCBI database is NP_002167.1; for the cDNA, accession number in the NCBI database is NM_002176.3.

According to various embodiments, the invention contemplates the use of a soluble IFN-β, an isolated IFN-β, a recombinant IFN-β, or a modified IFN-β e.g. by PEGylation or other half-life elongating moieties. According to one embodiment, the IFN-β is conjugated to a half life elongating moiety. For example, U.S. Pat. Nos. 8,557,232 and 7,670,595 (both incorporated herein by reference) disclose IFN-β derivatives, stabilized by fusion of an immunoglobulin Fc region, and U.S. Pat. No. 7,338,788 (incorporated herein by reference) discloses additional IFN-β variants and conjugates.

According to one embodiment, the invention contemplates the use of an active fragment of IFN-β i.e. a molecule comprising an IFN-β sequence or mimetics thereof capable of upregulating the activity of IRF7 although it does not comprise the full length protein.

According to one embodiment, IFN-β can be obtained commercially from e.g. Bayer Healthcare (under the brand name: BETASERON®), Biogen (under the brand name: AVONEX®), EMD Serono, Inc. or Pfizer (under the brand name: Rebif®), CinnaGen (under the brand name: CinnoVex®).

Additionally or alternatively, upregulating the anti-inflammatory response and/or treating an inflammation can be affected by expressing IFN-β in the subject.

Upregulation of IFN-β can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

Following is a list of agents capable of upregulating the expression level and/or activity of IFN-β.

An agent capable of upregulating expression of an IFN-β may be an exogenous polynucleotide sequence designed and constructed to express at least a functional portion of the IFN-β. Accordingly, the exogenous polynucleotide sequence may be a DNA or RNA sequence encoding an IFN-β molecule, capable of upregulating anti-inflammatory response.

The phrase “functional portion” as used herein refers to part of the IFN-β protein (i.e., a polypeptide) which exhibits functional properties of the enzyme such as binding to a substrate (e.g. to IRF7).

To express exogenous IFN-β in mammalian cells, a polynucleotide sequence encoding an IFN-β is preferably ligated into a nucleic acid construct suitable for mammalian cell expression. Such a nucleic acid construct includes a promoter sequence for directing transcription of the polynucleotide sequence in the cell in a constitutive or inducible manner.

Constitutive promoters suitable for use with some embodiments of the invention are promoter sequences which are active under most environmental conditions and most types of cells such as the cytomegalovirus (CMV) and Rous sarcoma virus (RSV). An inducible promoter suitable for use with some embodiments of the invention includes, for example, the tetracycline-inducible promoter (Zabala M, et al., Cancer Res. 2004, 64(8): 2799-804).

To express exogenous IFN-β in mammalian cells, a polynucleotide sequence encoding an IFN-β is preferably ligated into a nucleic acid construct suitable for mammalian cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of IFN-β mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding an IFN-β can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) as described in Liang C Y et al., 2004 (Arch Virol. 149: 51-60).

Recombinant viral vectors are useful for in vivo expression of IFN-β since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

It will be appreciated that upregulation of IFN-β can be also effected by administration of IFN-β-expressing cells into the individual.

IFN-β-expressing cells can be any suitable cells, such as lymphocyte or monocyte cells which are derived from the individuals and are transfected ex vivo with an expression vector containing the polynucleotide designed to express IFN-β as described hereinabove, as long as the cells are capable of entering the CNS.

Administration of the IFN-β-expressing cells of some embodiments of the invention can be effected using any suitable route for CNS administration Accordingly, suitable routes of administration include, but are not limited to, intracranial (IC) administration, intracerebroventricular (ICV) administration, intrathecal administration and/or intraparenchymal administration (into the spinal cord parenchyma), as described in detail hereinbelow.

IFN-β-expressing cells of some embodiments of the invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells [Williams D. Small is beautiful: microparticle and nanoparticle technology in medical devices. Med Device Technol. (1999) 10: 6-9; Desai, T. A. Microfabrication technology for pancreatic cell encapsulation. Expert Opin Biol Ther. (2002) 2: 633-46].

A “therapeutically effective amount” of IFN-β is an amount sufficient to upregulate the anti-inflammatory response and/or treat an inflammation in a subject (e.g. in a CNS of a subject) as discussed in detail hereinabove.

According to one embodiment, the therapeutically effective amount of IFN-β is an amount sufficient to upregulates the activity or expression of the transcription factor Interferon regulatory factor-7 (IRF7).

According to one embodiment, the therapeutically effective amount of IFN-β is an amount which downregulates (i.e. reduces by, for example, about 10%, 20%, 30%, 40%, 50%, 60%, 70%, 80%, 90% or 100% as compared to the gene expression in the absence of IFN-β) the expression of at least one pro-inflammatory associated gene. Exemplary pro-inflammatory associated genes include, but are not limited to, iNos, Tnfα, Il-1β, Cxcl1, Cxcl2 and Cxcl10.

According to one embodiment, the therapeutically effective amount of IFN-β is an amount sufficient to upregulate the expression of at least one anti-inflammatory associated gene. Exemplary anti-inflammatory associated genes include, but are not limited to, IL-10, MMR (CD206), DECTIN-1 and CD36.

According to one embodiment, the therapeutically effective amount of IFN-β is an amount sufficient to induce a M1-to-M2 (i.e. pro- to anti-inflammatory) phenotype conversion of a myeloid cell (e.g. microglia cell).

According to one embodiment, the therapeutically effective amount of IFN-β in mouse doses varies from 1-1000 ng per kg body weight per local administration, 10-500 ng per kg body weight per local administration, 100-250 ng per kg body weight per local administration. The doses can be effectively transformed to human uses by employing FDA conversion tables.

It will be appreciated that the doses may be greater provided that toxicity is avoided.

According to one embodiment, IFN-β is administered in a single dose or in multiple administrations, i.e., once, twice, three or more times daily or weekly over a period of time. In some cases, one or more doses may be given over a short period of time, including several hours to several days, alternatively, one or more doses may be given over an extended period of time, including, weeks, months or years.

IFN-β can be administered to a subject in need thereof using any methods or route known to one of ordinary skill in the art. According to one embodiment, IFN-β is administered using a local mode of administration (as described in further detail below). According to a specific embodiment, a mode of administration is selected such that the IFN-β of the invention does not need to cross the blood brain barrier.

As used herein, the term “local administration” refers to the site of inflammation or in close proximity to the site of inflammation (e.g. injury).

According to one embodiment of the invention, IFN-β is administered directly into the central nervous system.

According to some embodiments of the invention, administration into the CNS is effected by intracranial (IC) administration, intracerebroventricular (ICV) administration, intrathecal administration and/or intraparenchymal administration (i.e. intra CSF) delivery.

The phrase “intracranial (IC) administration” as used herein refers to administration into the brain parenchyma.

As used herein the phrase “intracerebroventricular (ICV) administration” refers to administration into the lateral ventricles of the brain.

As used herein the phrase “intrathecal administration” refers to administration into the cerebrospinal fluid or into the cisterna magna (also referred to as the cerebellomedullary cistern) of the brain of a subject. For example, intrathecal administration can be into the spinal canal (intrathecal space surrounding the spinal cord) such as near the subject's waist.

Methods of intracranial, intracerebroventricular and/or intrathecal administration are known in the art and are described, for example, in Pathan S A, et al. (2009) “CNS drug delivery systems: novel approaches.” Recent Pat. Drug Deliv. Formul. 3: 71-89; Geiger B M, et al. (2008) “Survivable Stereotaxic Surgery in Rodents.” J Vis Exp. 20, pii: 880. doi: 10.3791/880; Huang X, (2010) “Intracranial Orthotopic Allografting of MeduUoblastoma Cells in Immunocompromised Mice.” J Vis Exp. 44, pii: 2153. doi: 10.3791/2153; Alam M L et al. (2010 “Strategy for effective brain drug delivery”. Review. European J. of Pharmaceutical Sciences, 40: 385-403; Bakhshi S., et al. (1995) “Implantable pumps for drug delivery to the brain”. Journal of Neuro-Oncology 26:133-139; each of which is fully incorporated herein by reference.

For example, intracerebral delivery of the IFN-β of some embodiments of the invention into the parenchymal space of the brain can be achieved by directly injecting (using bolus or infusion) the IFN-β via an intrathecal catheter, or an implantable catheter essentially as described in Haugland and Sinkjaer, (1999) “Interfacing the body's own sensing receptors into neural prosthesis devices”. Technol. Health Care, 7: 393-399; Kennedy and Bakay, (1998) “Restoration of neural output from a paralyzed patient by a direct connection”. Neuroreport, 9: 1707-1711; each of which is fully incorporated herein by reference]. For example, the catheter can be implanted by surgery into the brain where it releases the IFN-β for a predetermined time period.

Intrabrain administration of IFN-β can be at a single injection, at a continuous infusion, or periodic administrations, and those of skills in the art are capable of designing a suitable treatment regime depending on the condition to be treated, and the subject to be treated.

According to some embodiments of the invention, the IC, ICV or intrathecal administration is performed by an injection or an infusion, using e.g., a needle, a syringe, a catheter, a pump, an implantable device (e.g., as is further described hereinunder) and/or any combination(s) thereof.

According to some embodiments of the invention, the IC, ICV or intrathecal administration is performed periodically.

Additionally or alternatively, the IFN-β of some embodiments of the invention may be administered using other routes of administration as long as the IFN-β can efficiently cross of the blood brain barrier.

According to one embodiment, IFN-β is administered by the trigeminal nerve and/or the olfactory nerve. Such nerve systems can provide a direct connection between the outside environment and the brain, thus providing advantageous delivery of a regulatory agent to the CNS, including brain, brain stem, and/or spinal cord. Methods for delivering agents to the CNS via the trigeminal nerve and/or the olfactory nerve can be found in, for example, WO 00/33813; WO 00/33814; and co-pending U.S. patent application No. 20130028874; all of which are incorporated herein by reference.

The IFN-β of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the IFN-β accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions of the invention include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

Pharmaceutical compositions suitable for use in context of some embodiments of the invention include compositions wherein the active ingredients are contained in an amount effective to achieve the intended purpose. More specifically, a therapeutically effective amount means an amount of active ingredients (IFN-β) effective to prevent, alleviate or ameliorate symptoms of a disorder (e.g., inflammation in the CNS) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Suitable models for CNS inflammation or injury are disclosed, for example, in Xiong et al., “Animal models of traumatic brain injury” Nat Rev Neurosci. (2013) 14(2): 128-142 (incorporated herein by reference) and are also available by e.g. Charles River Laboratories. Suitable models for spinal cord injury are discussed in Zhang et al., Neural Regen Res. 2014 Nov. 15; 9(22): 2008-2012 (incorporated herein by reference). Suitable models of focal and global cerebral ischemia (in small and large animal models) and discussed in Traystman ILAR J (2003) 44(2): 85-95 (incorporated herein by reference).

Toxicity and therapeutic efficacy of the active ingredients described herein can be determined by standard pharmaceutical procedures in vitro, in cell cultures or experimental animals (as mentioned above). The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide CNS levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Efficacy of treatment, i.e. reduction in inflammation of the CNS or disease treatment can be determined using any method known in the art. For example, reduction in inflammation can be determined e.g. by ultrasound, by MRI, by analysis of the cerebrospinal fluid (CSF) obtained by lumbar puncture (LP) and/or by blood tests testing specific markers [e.g. microglial activation (lbal), astrocytic response (GFAP), and/or neuronal loss (NeuN or Fluorojade for dying neurons] or measuring the levels of various pro-inflammatory cytokines (e.g. TNF-α and TGF-β). The test results can be compared to the same parameters in a healthy individual or to the test results of the subject prior to the treatment.

Likewise, reduction in injury or trauma to the CNS can be determined using imaging techniques, such as Positron Emission Tomography (PET), computerized tomography (CT) scan, MRI and/or ultrasound.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Animals

Adult male C57BL/6J, Cx3cr1^{GFP/+} (previously described in Jung S et al., Molecular and cellular biology (2000) 20: 4106-4114), and eGFP mice aged 8-10 weeks, or neonatal (P0-P1) C57BL/6J mice were used. Animals were supplied by the Animal Breeding Center of the Weizmann Institute of Science. All animals were handled according to the regulations formulated by the Institutional Animal Care and Use Committee (IACUC).

BM Radiation Chimeras

eGFP>WT BM chimeras were prepared by subjecting mice to lethal split-dose γ-irradiation (300 rad followed 48 hours later by 950 rad with head protection). After 1 day following the second irradiation, the mice were injected with 5×10⁶ bone marrow (BM) cells harvested from the hind limbs (tibia and femur) and forelimbs (humerus) of eGFP donor mice. BM cells were obtained by flushing the bones with Dulbecco's PBS under aseptic conditions, and then collected and washed by centrifugation (10 minutes, 1,250 rpm, 4° C.). After irradiation, mice were maintained on drinking water fortified with cyproxin for 1 week to limit infection by opportunistic pathogens. The percentage of chimerism was determined in the blood according to percentages of GFP expressing cells out of circulating monocytes (CD115). Using this protocol, an average of 90% chimerism was achieved.

Spinal Cord Injury (SCI)

The spinal cords of deeply anesthetized mice were exposed by laminectomy at T12, and contusive (200 kdynes) centralized injury was performed using the Infinite Horizon spinal cord impactor (Precision Systems), causing bilateral degeneration without complete penetration of the spinal cord. The animals were maintained on twice-daily bladder expression. Animals that were contused in a nonsymmetrical manner were excluded from the experimental analysis.

Intra-Parenchymal Injections

The spinal cords of deeply anesthetized mice were exposed 1 day following spinal cord injury and two injections of 1 μl PBS or IFN-β1 (800 ng/ml) were performed at the margins of the lesion site, in depth of 1.2 mm and injection rate of 250 nl/min.

Flow Cytometry Analysis and Sorting

Mice subjected to spinal cord injury were killed by an overdose of anaesthetic, and their spinal cords were prepared for flow cytometric analysis by perfusion with PBS via the left ventricle. The injured sites of spinal cords were dissected from individual mice (parenchymal segments of 0.5 mm from each side of the spinal cord lesion site), and tissues were homogenized using a software controlled sealed homogenization system [Dispomix; www(dot)biocellisolation(dot)com]. Cells were analyzed on a FACS-LSRII cytometer (BD Biosciences) using FlowJo software. Isotype controls were routinely used in intracellular experiments. All samples were filtered through an 80 μm nylon mesh and blocked with Fc-block CD16/32 (BD Biosciences). Next, samples were stained using the following antibodies: FITC-conjugated CD11b, Percp Cy5.5-conjugated Ly6C, and PE-conjugated CD115 (all purchased from eBioscience); PE-conjugated isotype control IgG2b(k), Pacific Blue-conjugated CD45.2, and APC-conjugated Ly6G (all purchased from Biolegend); PE-conjugated IL-10 (purchased from BD Biosciences).

In sorting experiments, 500 microglia and mo-MΦ cells derived from eGFP>WT chimeras were sorted using SORP-FACS sorter (BD Biosciences) into 25 μl of lysis buffer at different time points following SCI. RNA was extracted from sorted cells, DNA libraries were produced, and sequencing was conducted, as described below.

Mixed Brain Glial and Primary Microglial Cultures

Brains from neonatal (POP1) C57BL/6J mice were stripped of their meninges and choroid plexus in Leibovitz-15 medium (Biological Industries, Beit Ha-Emek, Israel). After trypsinization (0.5% trypsin, 10 min, 37° C., 5% CO2), the tissue was triturated. The cell suspension was washed in DMEM supplemented with 10% FCS, 1 mM L-glutamine, 1 mM sodium pyruvate, 100 U/ml penicillin, and 100 μg/ml streptomycin. The mixed brain glial cells were cultured at 37° C., 5% CO2 in 75-cm² Falcon tissue-culture flasks (BD Biosciences) coated with poly-D-lysine (PDL) (10 μ/ml; Sigma-Aldrich, Rehovot) for 5 hours, then washed thoroughly with sterile distilled water. The medium was replaced after 24 hours in culture and every 2^{nd} day thereafter, for a total culture period of 10 to 14 days. Microglia were shaken off the primary mixed brain glial cell cultures (170 rpm, 37° C., 6 hours) with maximum yields between days 10 and 14, and seeded (10⁵ cells/ml) onto 24-well plates (1 ml/well; Corning, Corning, N.Y.) pretreated with poly-d-lysine. Cells were grown in culture medium for microglia [RPMI-1640 medium (Sigma-Aldrich, Rehovot) supplemented with 10% FCS, 1 mM L-glutamine, 1 mM sodium pyruvate, 100 U/ml penicillin, and 100 μg/ml streptomycin]. After seeding, newborn-derived microglia (NB-Mg) were left untreated, stimulated with 100 ng/ml LPS (E. Coli 055:B5, Sigma-Aldrich, Rehovot) for 4 hours, or stimulated with 100 ng/ml LPS for 20 hours, washed with warm culture medium and re-challenged with 100 ng/ml LPS for 4 hours.

Bone Marrow Macrophage Culture

Bone marrow progenitors were harvested from C57BL/6J mice and cultured for 7 days on Petri dishes (0.5×10⁶ cells/ml) in RPMI-1640 supplemented with 10% FCS, 1 mM L-glutamine [1 mM sodium pyruvate, 100 U/ml penicillin, 100 μg/ml streptomycin, and 50 ng/ml M-CSF (Peprotech)]. At day 7, cells were detached with cold PBS and replated on 24-well tissue-culture plates (0.5×10⁶ cells/ml; Corning, Corning, N.Y.). On day 8, bone-marrow derived macrophages (BM-MΦ) were either left untreated, stimulated with 100 ng/ml LPS (E. Coli 055:B5, Sigma-Aldrich, Rehovot) for 4 hours or stimulated with 100 ng/ml LPS for 20 hours, washed with warm culture medium and re-challenged with 100 ng/ml LPS for 4 hours.

Activation Reagents

BM-MΦ and NB-Mg were preconditioned for 20 hours with 100 ng/ml TGF-β1 (Peprotech), 10 ng/ml IL-4 (Peprotech), 10 ng/ml IL-13 (Peprotech) or 100 ng/ml TGF-β2 (Peprotech), washed with culture medium, and stimulated for 20 hours with 100 ng/ml LPS, washed again, and then re-challenged for 4 hours with 100 ng/ml LPS. Cells were then washed with PBS, and total RNA was extracted. For induction of Irf7 expression, LPS-polarized NB-Mg were stimulated with 1000 U/ml IFN-β1 (PBL Interferon Source) for 1 hour prior to an additional 4 hours LPS re-challenge (100 ng/ml).

RNA Interference

BM-MΦ were transfected with siRNA directed against Irf7 or scrambled siRNA (Dharmacon) with Lipofectamine 2000 (Invitrogen), according to the manufacturer's instructions. In brief, siRNA and Lipofectamine were diluted in Opti-MEMI Reduced Serum Medium (Invitrogen), mixed, incubated for 20 minutes at room temperature and added to the BM-MΦ cultures. The cells were incubated with the transfection mixture for 5 hours, and the BM-MΦ were stimulated as described above. The IRF7 siRNA consisted of four pooled 19-nucleotide duplexes. The sequences of the four duplexes were CCAACAGUCUCUACGAAGA (SEQ ID NO: 1), CCAGAUGCGUGUUCCUGUA (SEQ ID NO: 2), GAGCGAAGAGGCUGGAAGA (SEQ ID NO: 3), and GCCCUCUGCUUUCUAGUGA (SEQ ID NO: 4).

Gene Expression Analysis

RT-qPCR

NB-Mg and BM-MΦ were stimulated as described above and washed with PBS. Total RNA of in vitro cultured or in vivo sorted cells following SCI was extracted with the miRNeasy kit according to the manufacturer's instructions (Qiagen). For RNA extraction from the spinal cord, the excised tissues were homogenized in Tri-reagent (Sigma Aldrich) and RNA was extracted with the RNeasy kit according to the manufacturer's instructions (Qiagen). RNA was reverse-transcribed with the high capacity cDNA reverse transcription kit (Applied Biosystems), amplified using SYBR green I Master Mix (Roche) and detected by the LightCycler 480 (Roche) in duplicates. Results were normalized to the expression of the housekeeping gene, Peptidylprolyl Isomerase A (PPIA), and then expressed as fold up-regulation with respect to the control sample. For a list of the primers that were used in this study refer to Table 1 below.

TABLE 1
RT-qPCR primers

| Primer | Forward sequence | Reverse sequence |
|---|---|---|
| Ppia | AGC ATA CAG GTC CTG GCA TCT T [symbol] | CAA AGA CCA CAT GCT TGC CAT C [symbol] |
| | (SEQ ID NO: 5) | (SEQ ID NO: 6) |
| Irf7 | CTT CAG CAC TTT CTT CCG AGA (SEQ [symbol] | TGT AGT GTG GTG ACC CTT GC (S [symbol] |
| | NO: 7) | ID NO: 8) |
| Lgals3 | GGT GGA GCA CTA ATC AGG AAA (S [symbol] | CGG ATA TCC TTG AGG GTT TG (S [symbol] |
| | ID NO: 9) | ID NO: 10) |
| Ccl9 | CCC ATG TGA AAC ATT TCA ATT [symbol] | TGG GCC CAG ATC ACA CAT (SEQ [symbol] |
| | (SEQ ID NO: 11) | NO: 12) |
| Anxa1 | CCA AGA GGA CCA ATG CTC AG (SEQ [symbol] | GGC TTT TCT CAA GAC TTC ATC |
| | NO: 13) | (SEQ ID NO: 14) |
| Tnf | CCC TCA CAC TCA GAT CAT CTT [symbol] | GCT ACG ACG TGG GCT ACA G (SEQ [symbol] |
| | (SEQ ID NO: 15) | NO: 16) |
| Cxcl1 | GAC TCC AGC CAC ACT CCA AC (SEQ [symbol] | TGA CAG CGC AGC TCA TTG (SEQ [symbol] |
| | NO: 17) | NO: 18) |
| Cxcl2 | AAA ATC ATC CAA AAG ATA CTG A [symbol] | CTT TGG TTC TTC CGT TGA GG (SEQ [symbol] |
| | AA (SEQ ID NO: 19) | NO: 20) |
| Il-10 | TTT GAA TTC CCT GGG TGA GAA (S [symbol] | GGA GAA ATC GAT GAC AGC GC (S [symbol] |
| | ID NO: 21) | ID NO: 22) |
| Nos2 | CTT TGC CAC GGA CGA GAC (SEQ ID N [symbol] | TCA TTG TAC TCT GAG GGC TGA |
| | 23) | (SEQ ID NO: 24) |
| Pmepa1 | CAA ATC GTG GTC ATC GTG GT (SEQ [symbol] | TTC CAC CTG ACA CCG TAC TC (S [symbol] |
| | NO: 25) | ID NO: 26) |
| Il-6 | TGCAAGAGACTTCCATCCAGTTG (S [symbol] | TAAGCCTCCGACTTGTCAAGTGGT |
| | ID NO: 27) | (SEQ ID NO: 28) |
| Il-1b | ACCTGT CCT GTGTAATGA AAG A [symbol] | TGG GTATTG CTT GGG ATC CA (SEQ [symbol] |
| | (SEQ ID NO: 29) | NO: 30) |
| Cxcl10 | AACTGCATCCATATCGATGAC (SEQ [symbol] | GTGGCAATGATCTCAACAC (SEQ [symbol] |
| | NO: 31) | NO: 32) |
| Tgfbr1 | CGA GAG GCA GAG ATT TAT CAG (S [symbol] | ATG TCC CAT TGT CTT TGT TGT C (S [symbol] |
| | ID NO: 33) | ID NO: 34) |
| Tgfbr2 | TTT CGG AAG AAT ACA CCA CCA (S [symbol] | ATG ATG ACA GCT ATG GCA ATC |
| | ID NO: 35) | (SEQ ID NO: 36) |

[symbol]

indicates data missing or illegible when filed

RNA Sequencing

NB-Mg and BM-MΦ were harvested at different time points following TGF-β1 or LPS preconditioning. Total RNA was extracted with the miRNeasy kit according to the manufacturer's instructions (Qiagen). RNA concentrations of the samples were measured using Qubit HS RNA kit (Invitrogen), and quality was tested using TapeStation HS RNA. Total RNA (100 ng) was heat-fragmented at 94° C. for 5 minutes into fragments with an average size of 300 nucleotides (NEBNext Magnesium RNA Fragmentation Module) and the 3′ polyadenylated fragments were enriched by selection on poly dT beads (Dynabeads, Invitrogen). The RNA was reverse transcribed to cDNA using smart-scribe RT kit (Clontech). Illumina compatible adaptors were added using NEB Quick ligase, and the DNA library was amplified by PCR using P5 and P7 Illumina compatible primers (IDT). DNA concentration was measured by Qubit DNA HS, and the quality of the library was analyzed by Tapestation (Agilent). DNA libraries were sequenced on Illumina HiSeq-1500 with average of 5.8 million aligned reads per sample.

Pre-Processing of RNA-Seq Data

All reads were aligned to the mouse reference genome (NCBI 37, MM9) using the TopHat aligner [as described in Trapnell C. et al., Bioinformatics (2009) 25: 1105-1111]. The raw expression levels of the genes were calculated using Scripture [as described in Guttman M. et al., Nature biotechnology (2010) 28: 503-510], an ab-initio software for transcriptome reconstruction. Normalization was performed using DESeq [as described in Anders S. and Huber W, Genome biology (2010) 11: R106], a method based on the negative binomial distribution, with variance and mean linked by local regression. To analyze genes expressed by NB-Mg and BM-MΦ along the kinetics of TGF-β1 exposure, those genes that were expressed at a threshold greater than 30 (relative to t=0) on at least one time point along the time course were identified, and among them, only those that showed two-fold or greater change in at least one time point relative to others along the kinetics were selected. To analyze genes expressed by sorted microglia and mo-MΦ along the kinetics of following SCI, those genes that were expressed at a threshold greater than 10 (relative to t=0) on at least one time point along the time course were identified. For further analysis, genes were categorized into functional groups using PANTHER database of gene ontology [as described in Mi H. et al., Nature protocols (2013) 8: 1551-1566).

K-means clustering—Two-fold changed genes were clustered by partition of n observations to k clusters in which each observation is assigned to the cluster with the nearest mean. The next input, k=20 and a table log 2 data of effect X₍ₜ₌ₙ₎−X₍ₜ₌₀₎ and a column of X₍ₜ₋₀₎ was used. Clusters were manually reordered.

Chromatin Immunoprecipitation (ChIP)-Seq

Whole genome Irf7 binding profiles were obtained using high-throughput chromatin immunoprecipitation (HT-ChIP) as previously described [Garber M. et al., Molecular cell (2012) 47: 810-822]. Briefly, GM-CSF treated bone-marrow derived dendritic cells were collected following 2 hours of LPS treatment or untreated control. Cells were cross-linked with formaldehyde, lysed, and chromatin was fragmented by sonication. Irf7-DNA complexes were immunoprecipitated using anti-Irf7 antibody (B ethyl laboratories). After thorough washes, reverse cross-linking, and RNAse and Proteinase K treatment, a sequencing library was generated, followed by Illumina sequencing HiSeq-1500 (50 base, SR). Sequenced data reads were aligned to the mouse reference genome NCBI 37 MM9 using bowtie version 4.1.2. Bowtie alignments were processed by Scripture (as previously described in Guttman et al, 2010, supra) to obtain significantly expressed transcripts for each time course. Data were filtered by peak intensity of 40.

Statistical Analysis

Data were analyzed using Student's t-test to compare between two groups. One-way or two-way ANOVA tests were used to compare several groups; the Bonferroni posttest (p=0.05) was used for follow-up pairwise comparison of groups. Kolmogorov-Smirnov test was used to compare distributions. Hypergeometric distribution test was used to compare observed and expected gene lists size. The specific tests used to analyze each set of experiments are indicated in the figure legends. The results are presented as mean±standard error mean (SEM). *p<0.05, **p<0.01, ***p<0.001.

### Example 1

### M1-to-M2 Phenotype Switch of Newborn Microglia is Impaired by Long Exposure to TGF-β1

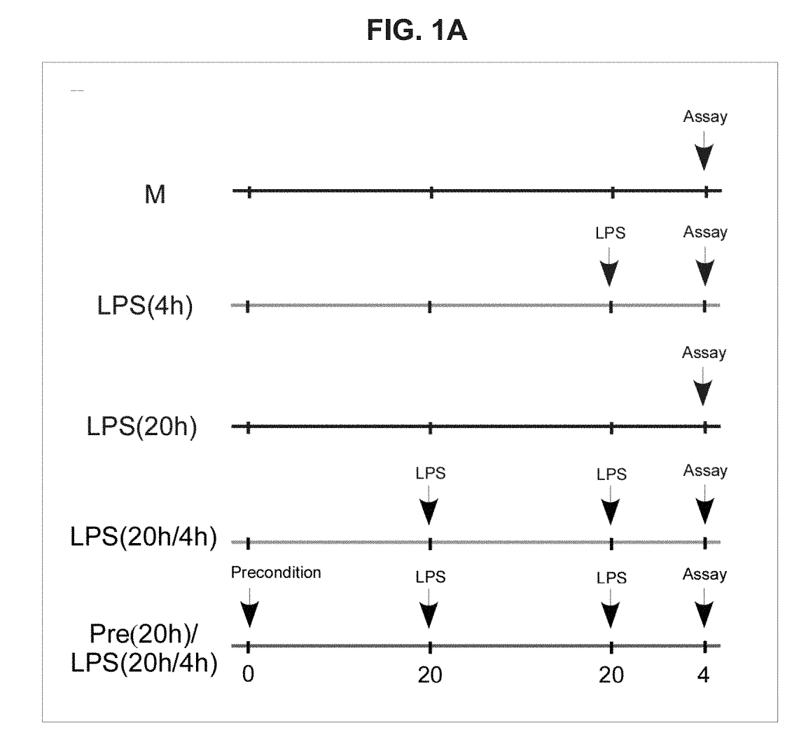

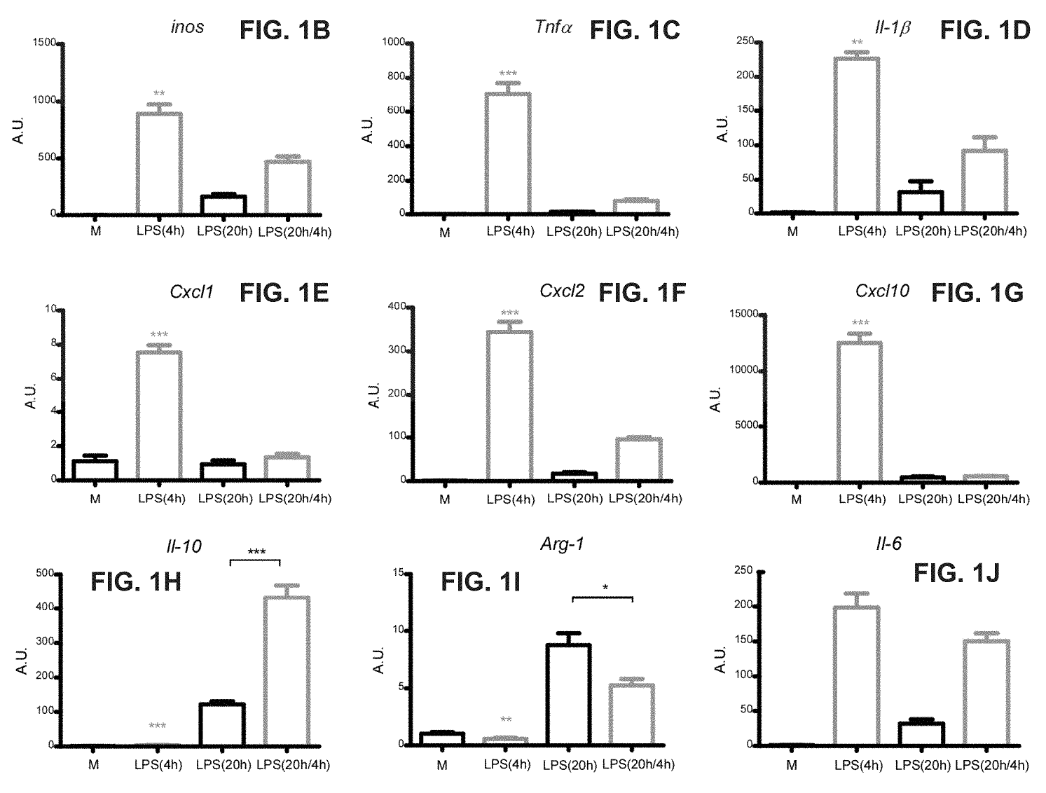

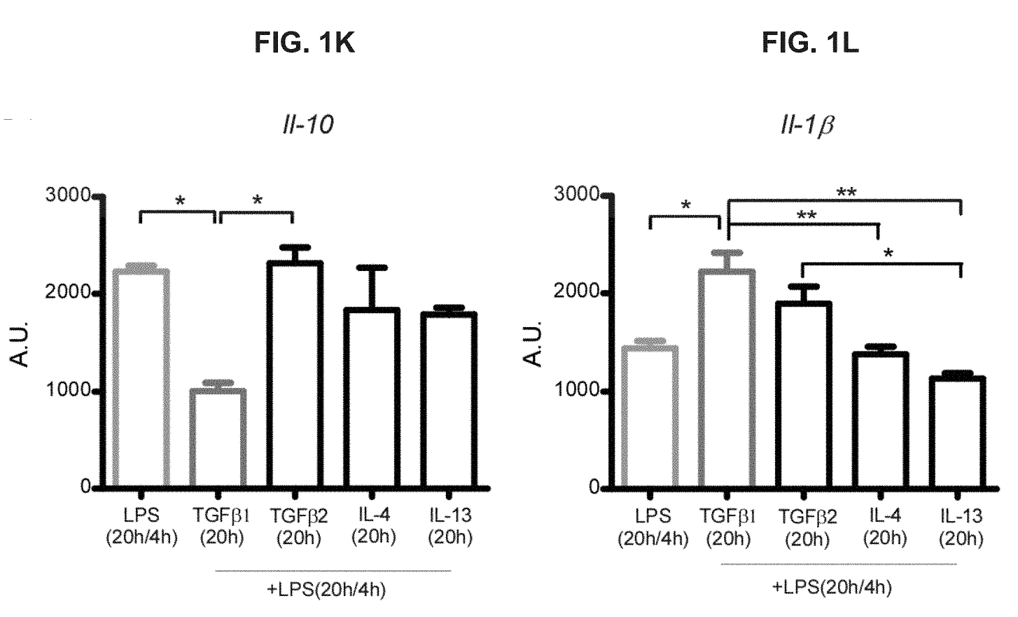

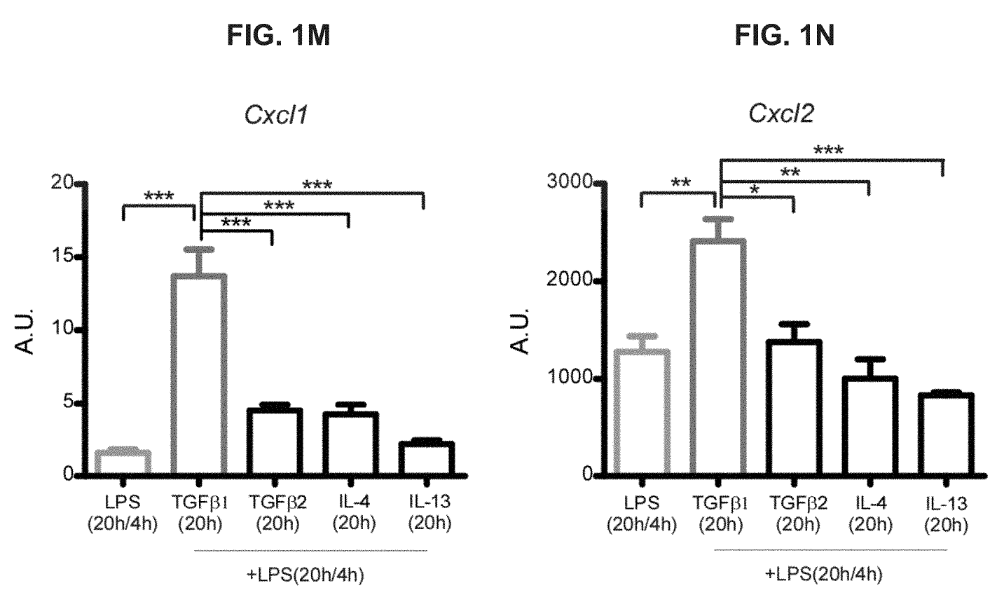

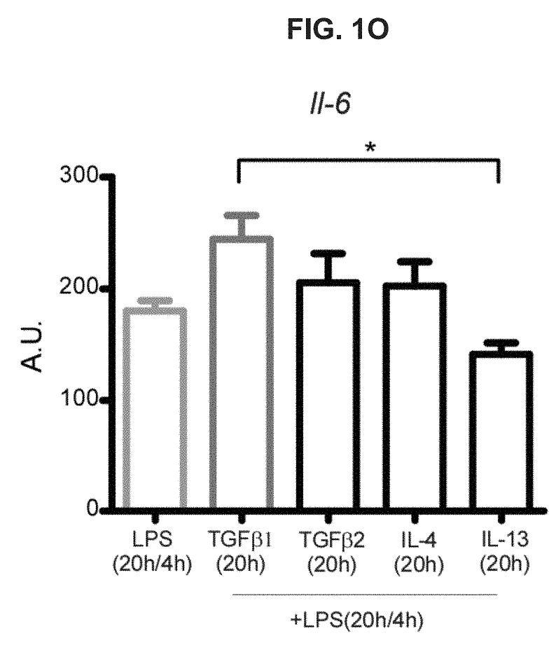

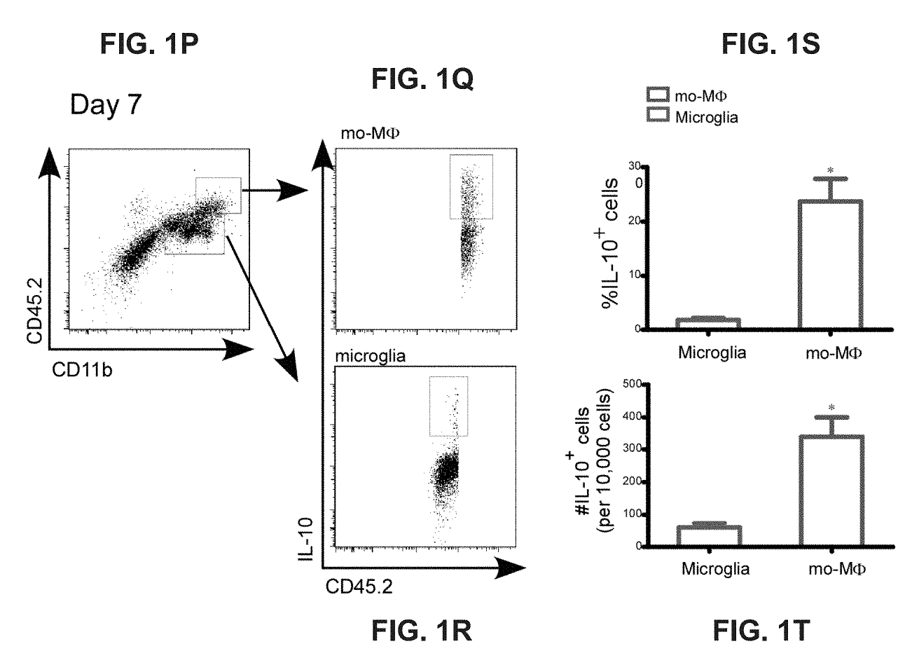

The present inventors' hypothesis was that although microglia differ in their origin from monocyte-derived macrophages (mo-MΦ), their response under pathological conditions within the central nervous system (CNS), is dictated to a large extent by their microenvironment. To test this hypothesis, the ability of newborn-derived microglia (NB-Mg) to undergo M1-to-M2 phenotype switch was first assessed. To this end, an established ex vivo model of macrophage polarization previously described by Porta [Porta C. et al., Proceedings of the National Academy of Sciences of the United States of America (2009) 106: 14978-14983] was adopted, in which M1 polarization, which is known to be induced by brief exposure to lipopolysaccharide (LPS, 4 hours), is inhibited as a result of extended LPS pre-exposure (20 hours). Under such conditions, the cells switch to an M2-like (anti-inflammatory) phenotype, and remain unresponsive to further LPS challenge. Using this ex vivo assay, the response of NB-Mg following 4 hours LPS challenge was compared to their response to such a challenge following a long (20 hours) pre-exposure to LPS (FIG. 1A). Cells were harvested, and total RNA was extracted to determine expression of characteristic pro- and anti-inflammatory genes. The gene expression profile of the treated NB-Mg showed their ability to undergo M1-to-M2 phenotype switch following long LPS pre-exposure, which was highly similar to the previously documented monocyte/macrophage M2-polarization phenotype (Porta et al., (2009), supra). Specifically, iNos, Tnfα, Il-1β, Cxcl1, Cxcl2 and Cxcl10, M1-associated pro-inflammatory genes involved in CNS inflammation and neurodegeneration, were down-regulated in LPS-tolerant cells, and barely induced following 4 hours LPS re-challenge (FIGS. 1B-J). Under the same experimental conditions, the prototype anti-inflammatory cytokine, Il-10, was induced rather than suppressed in the LPS-tolerant NB-Mg, and further elevated following re-challenge (FIG. 1H). These results indicate that NB-Mg, similarly to macrophages, have an inherent capacity to switch from M1 to M2 phenotype ex vivo under prolonged inflammatory conditions (e.g. long exposure to LPS).

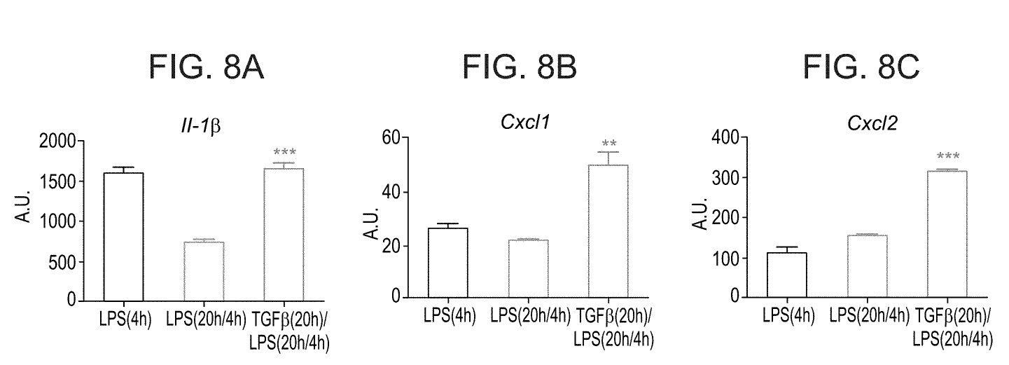

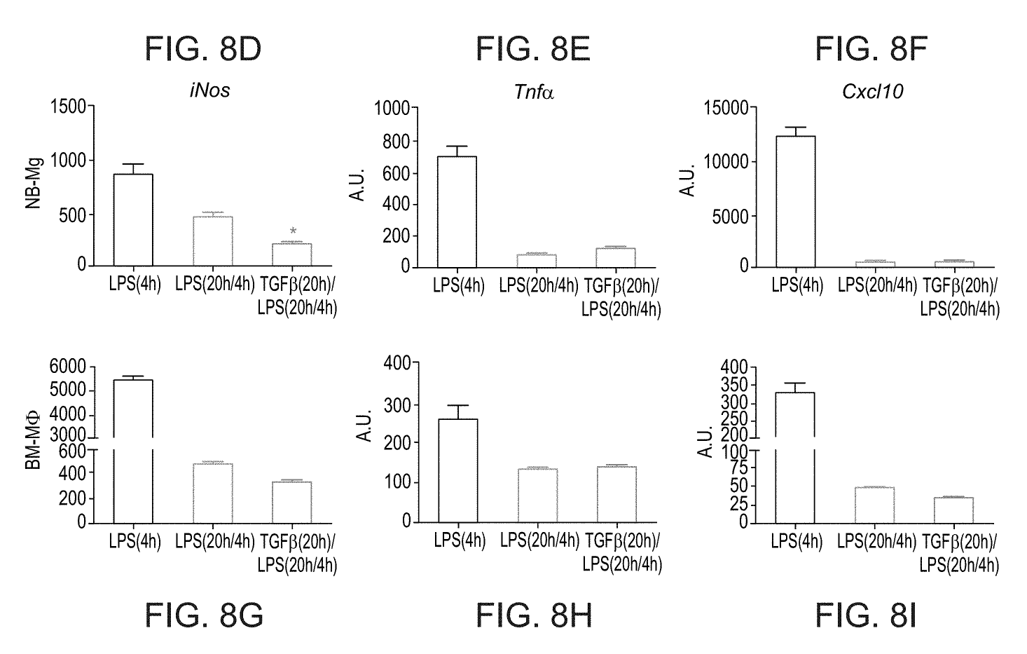

Next, NB-Mg were exposed, prior to LPS treatment, to factors prevalent within the CNS microenvironment, and their subsequent ability to undergo M1-to-M2 phenotype switch was examined. The same LPS tolerance model was used, but this time, the cells were first exposed to anti-inflammatory factors, such as TGF-β1, TGF-02, IL-4 and IL-13, and only then to LPS (FIG. 1A, bottom). The inventors' premise was that long exposure to such anti-inflammatory factors would create a form of tolerance to the tested anti-inflammatory cytokines, and would imprint the inability to switch from M1-to-M2 phenotype during the subsequent long LPS incubation. Of the tested factors, only incubation with TGF-β1 for 20 hours, before the subsequent exposure to LPS tolerance conditions, prevented the LPS-induced polarization towards M2-like phenotype with respect to expression of key characteristic cytokines; thus, the cells exposed to TGF-β1 prior to the LPS tolerance, showed down-regulation of Il-10 expression, and increased expression of the characteristic pro-inflammatory cytokines, including Il1β, Il-6, Cxcl1, and Cxcl2 (FIGS. 1K-O). Importantly, the pro-inflammatory bias caused by the extended pre-exposure to TGF-β1, prior to the LPS, was not restricted to microglia; a similar effect was observed when bone-marrow derived macrophages (BM-MΦ) were tested under the same experimental paradigm (FIGS. 8A-C). Notably, not all pro-inflammatory genes were affected; the expression of some genes, such as iNos, Tnfα and Cxcl10, was not affected by TGF-β1 pre-exposure in either NB-Mg or BM-MΦ (FIGS. 8D-I). The low Il-10 expression level in NB-Mg following TGF-β1 pre-exposure was reminiscent of adult microglial incompetence to secrete IL-10 in response to acute spinal cord injury (SCI), compared to high expression levels of this cytokine by mo-MΦ at the crucial phase of the repair process, day 7 (FIGS. 1P-T). Collectively, these results support the hypothesis that a TGF-β1-enriched microenvironment, to which adult microglia are exposed prior to CNS injury, impairs their ability to acquire an inflammation-resolving phenotype and to convert into M2-like cells under severe injurious conditions; in contrast, the blood-derived monocytes are freshly recruited, and thus have no experience of TGF-β1 pre-exposure.

### Example 2

### Long Exposure to TGF-β1 Activates a Robust Gene Expression Program in Myeloid Cells, with Implications to CNS Pathological Conditions

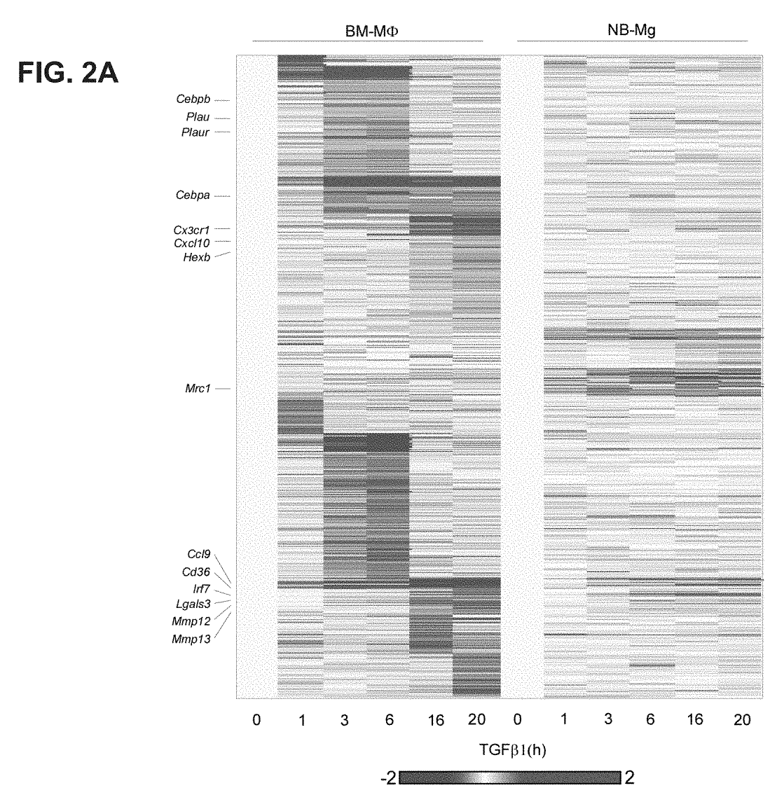

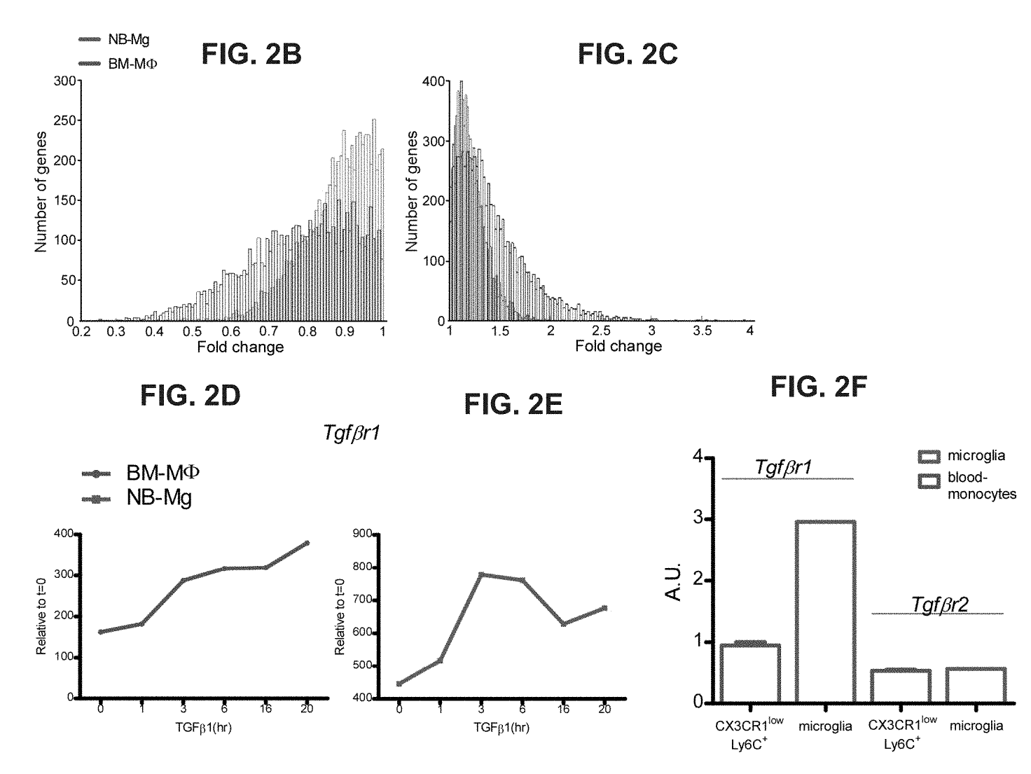

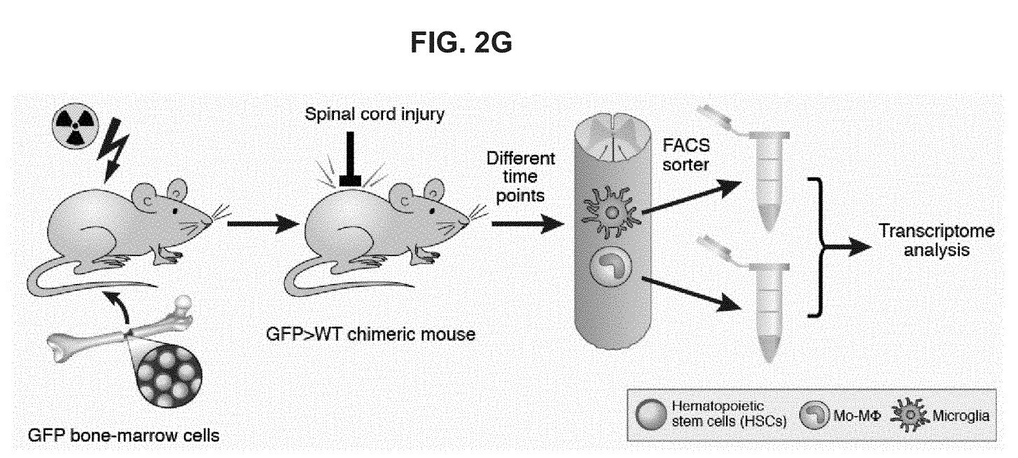

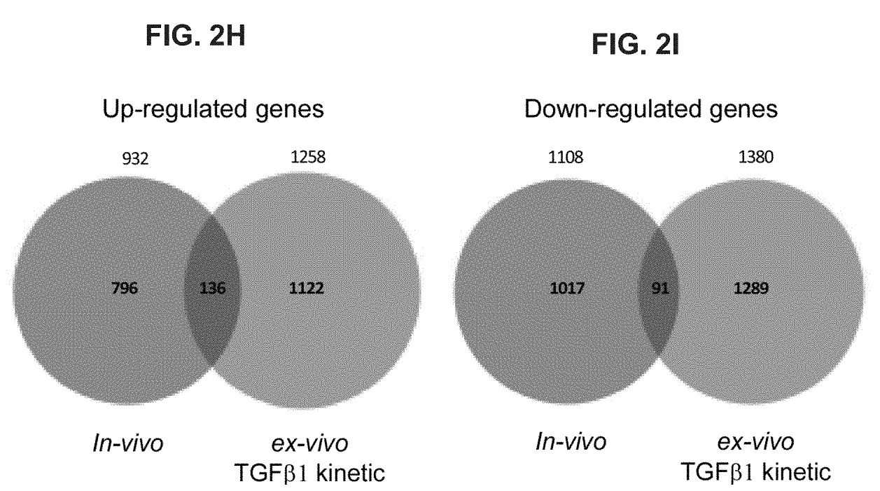

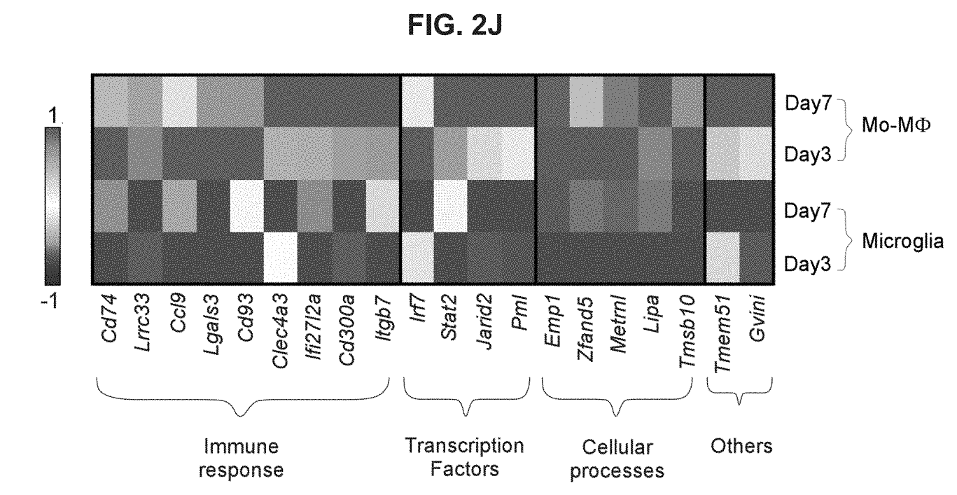

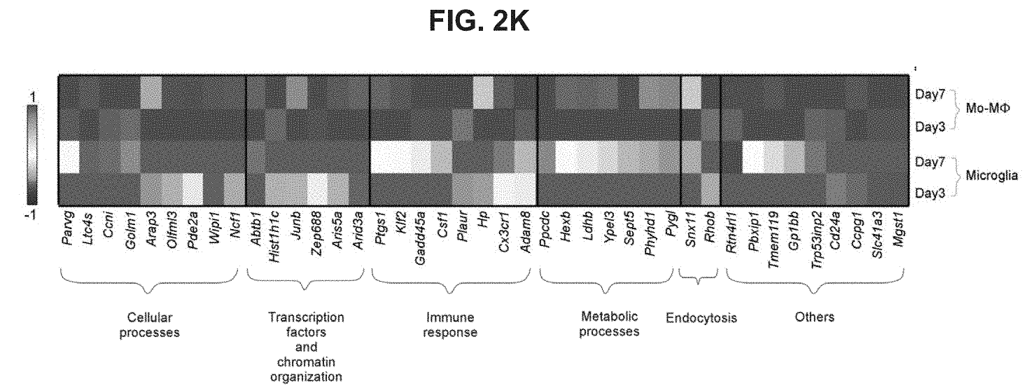

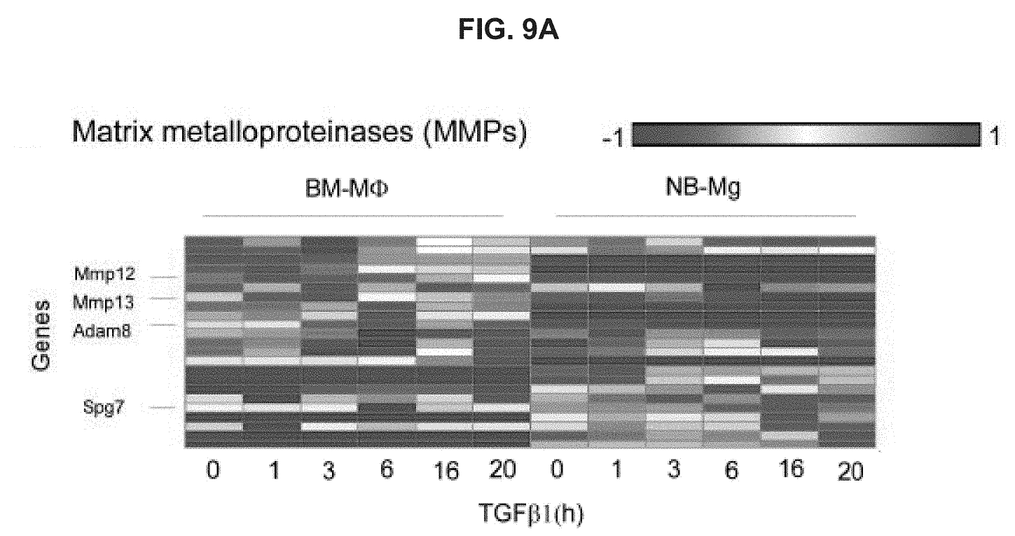

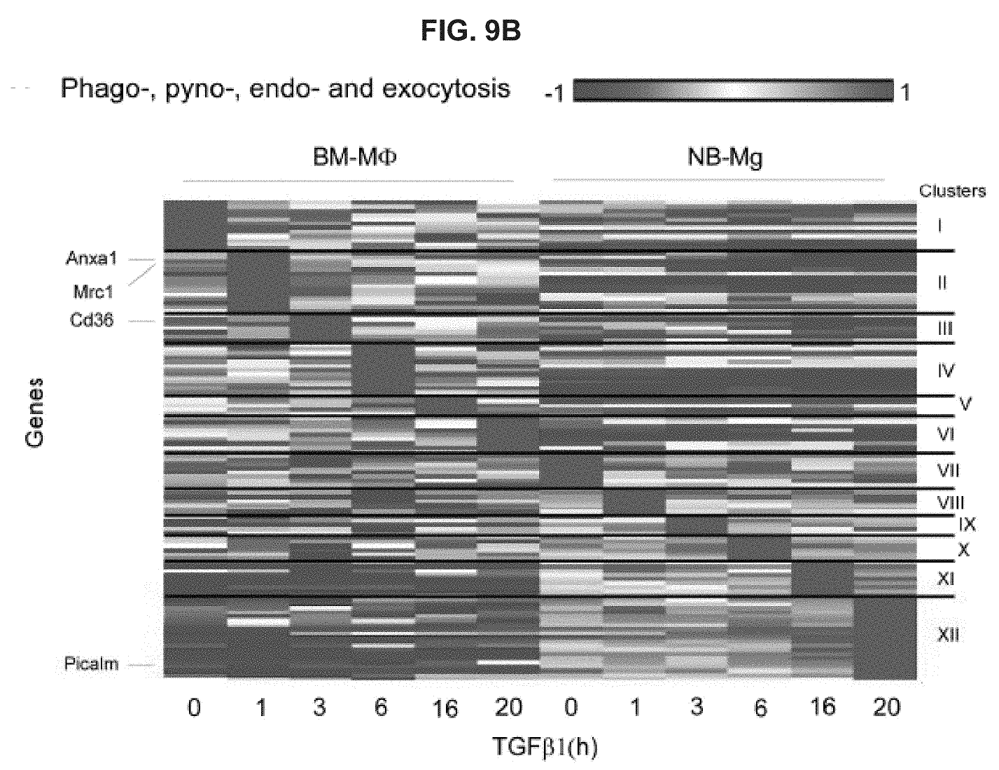

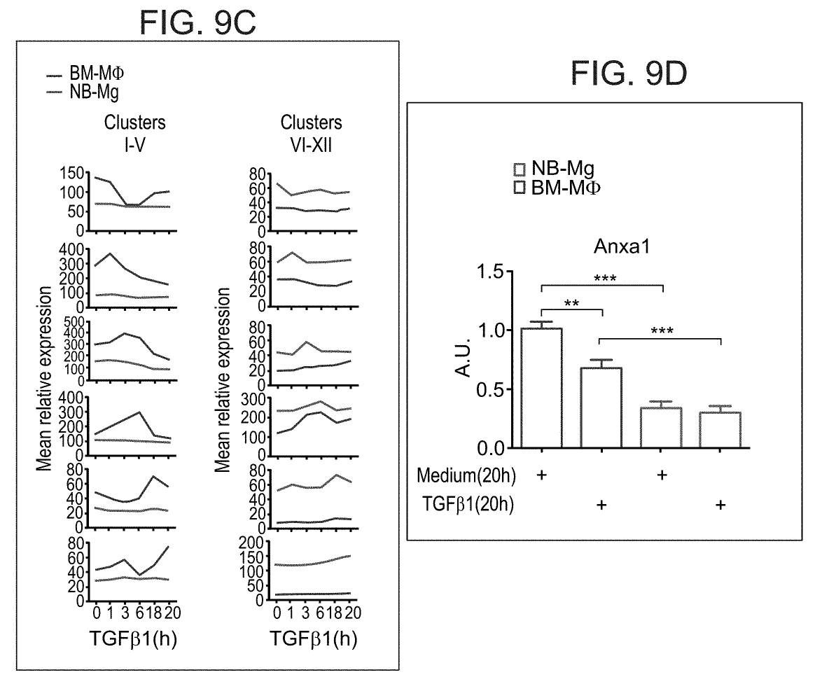

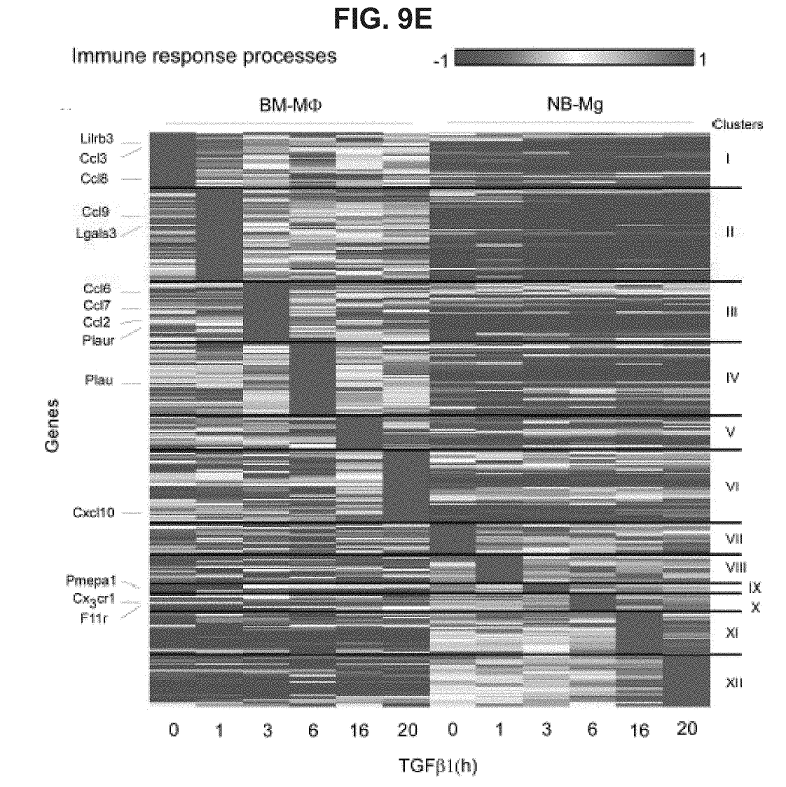

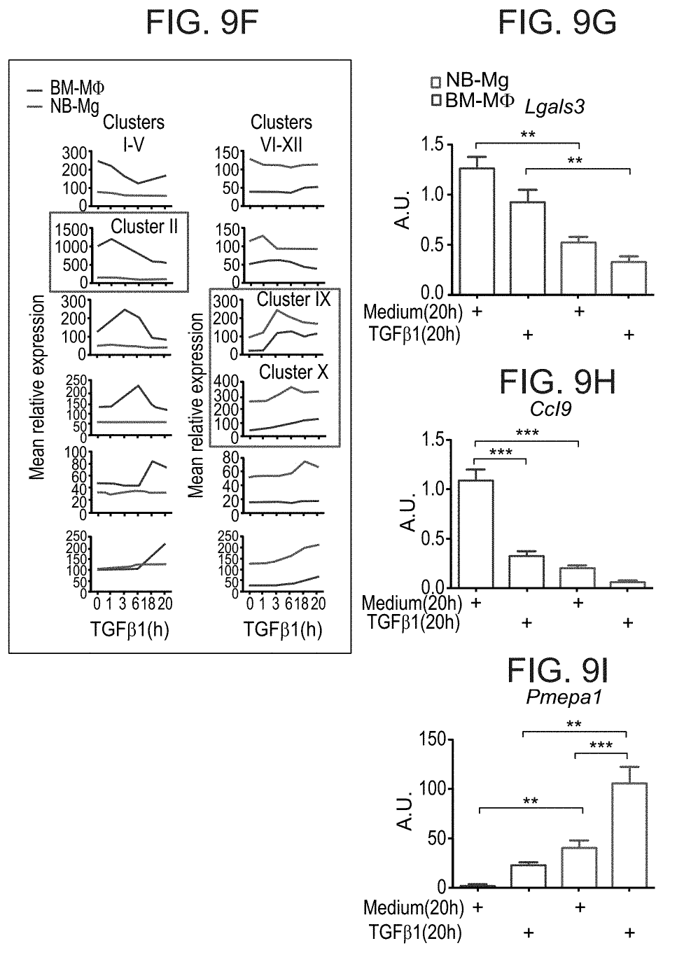

To understand the molecular events elicited by long exposure to TGF-β1, genome-wide expression profiles were measured using RNA-Seq of both BM-MΦ and NB-Mg along the time course of ex vivo TGF-β1 exposure. Globally, 2,721 and 642 genes showed expression changes (2 fold change; up or down) in response to TGF-β1 in BM-MΦ and NB-Mg, respectively (FIG. 2A). Notably, TGF-β1 induced a response that was similar in terms of gene expression pattern in both cell types, although they are of different developmental origin, BM vs. yolk sac, respectively (FIG. 2A). BM-MΦ exhibited higher responsiveness to TGF-β1 treatment compared to NB-Mg, reflected by the higher number of changed genes and the intensity of their change (p<10⁻⁵) (FIGS. 2B-C). The overall dynamic change of myeloid cell gene expression following long exposure to TGF-β1 revealed global effects of TGF-β1 on expression of genes involved in tissue repair processes (FIGS. 9A-I), as well as on up-regulation of its own receptor, Tgfbr1 (FIGS. 2D-E). To test the relevance of the observed effect of preconditioning with TGF-β1, but not with TGF-β2, on the M1-to-M2 switch ex vivo (FIGS. 1K-O) to the in vivo situation in adult CNS, the expression levels of TGF-β receptors by adult microglia and blood monocytes was examined. Adult microglia and blood monocytes were isolated from CX3CR1^{GFP/+} mice and were analyzed for their receptor levels. As illustrated in FIG. 2F, it was shown that resident adult microglia expressed higher levels of Tgfbr1 compared to blood-derived cells, whereas levels of Tgfbr2 were similar in both cell types (FIG. 2F). The higher expression of Tgfbr1 on adult microglia strengthened the potential relevance of TGF-β1 to the fate of these resident myeloid cells.

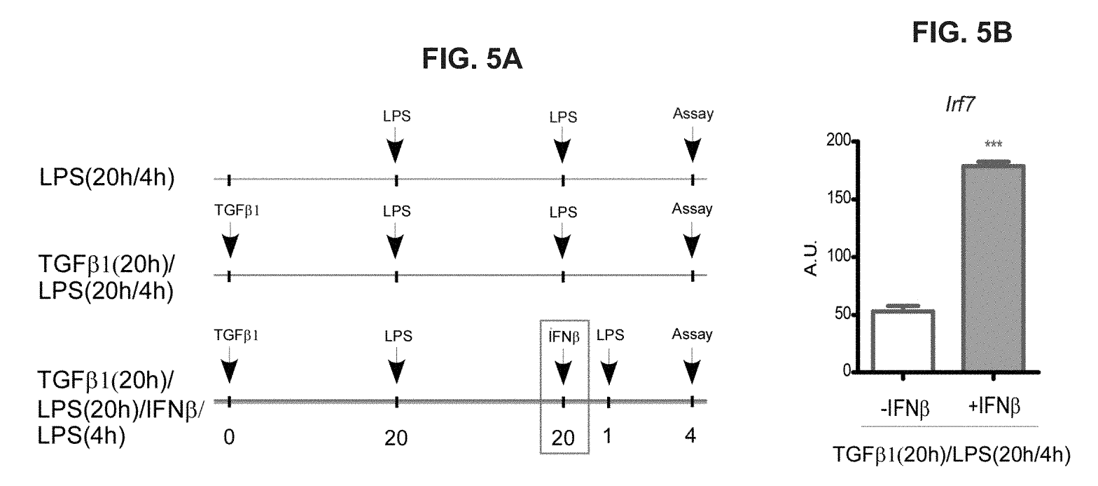

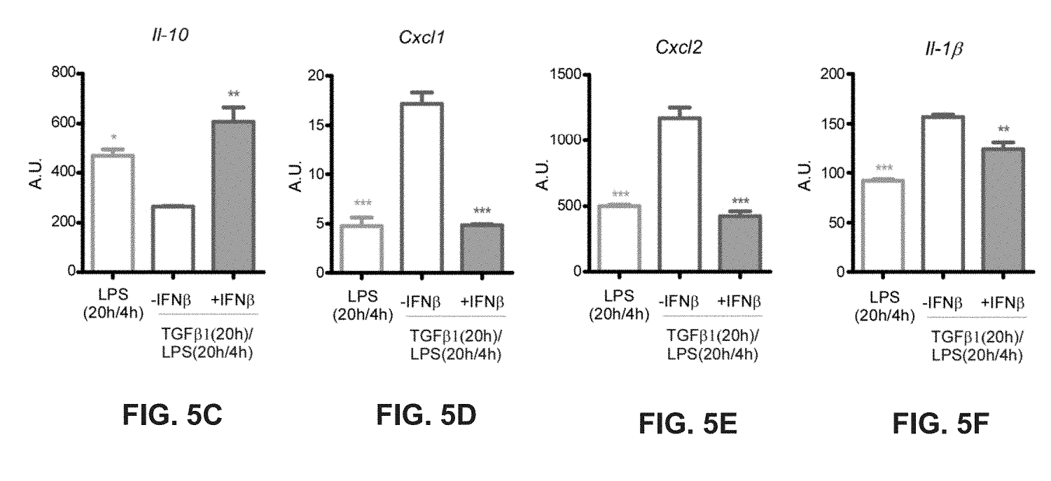

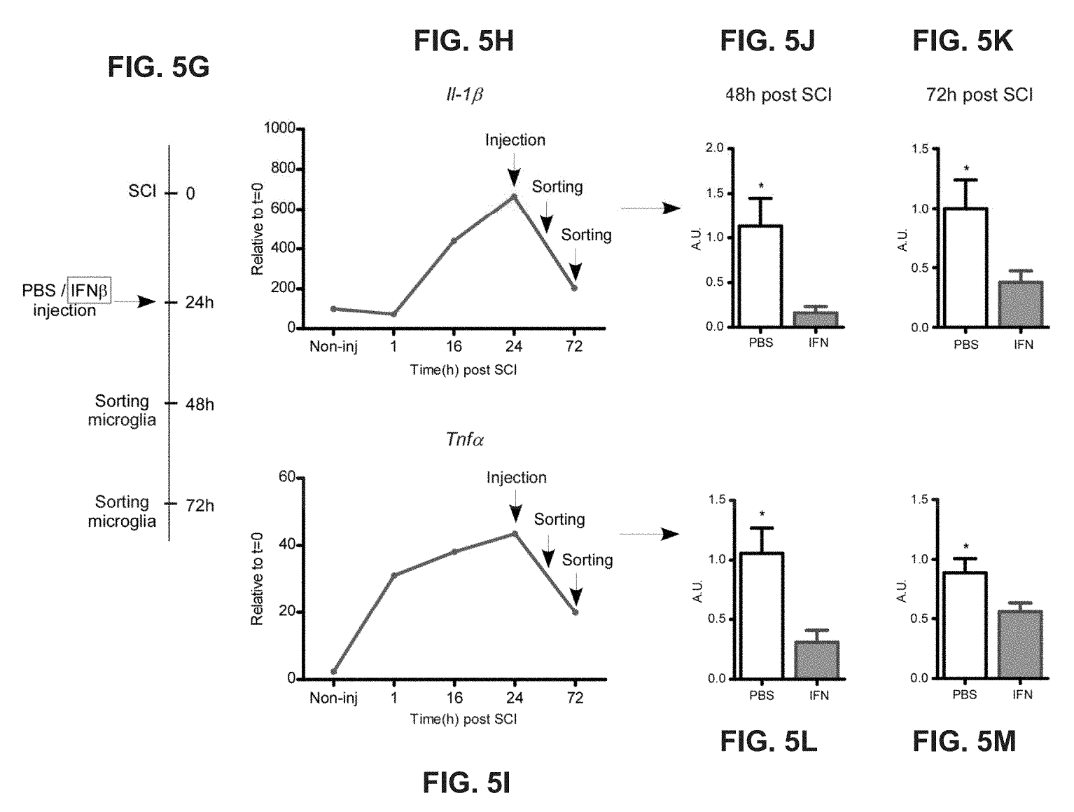

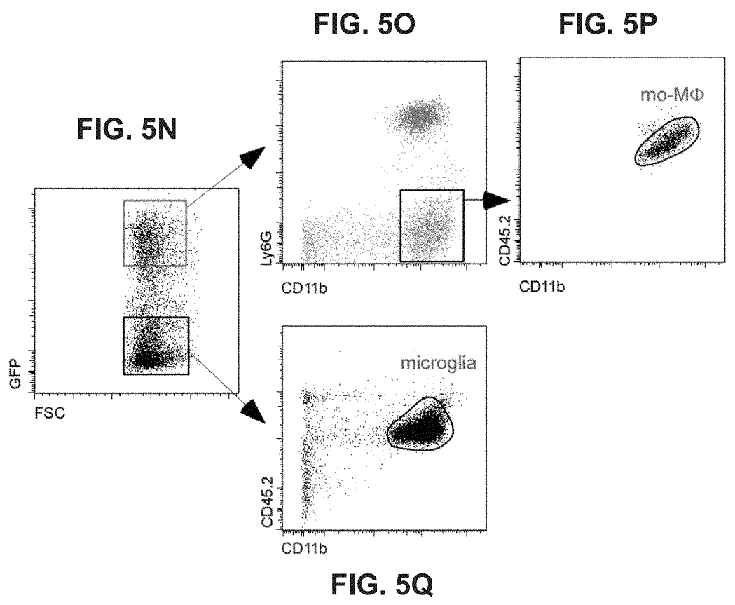

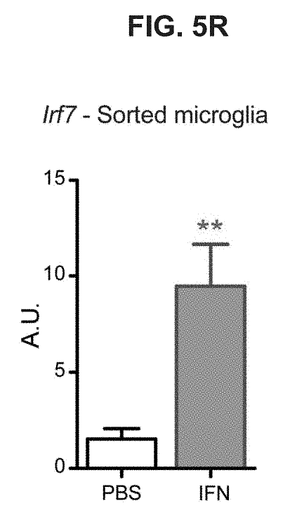

Since previous data showed that microglia and infiltrating mo-MΦ have distinct inflammation-resolving phenotypes following SCI [Shechter R. et al., PLoS medicine (2009) 6: e1000113; and Schechter (2013) supra], the activated resident microglia and the infiltrating mo-MΦ were isolated from the injured spinal cord, and their global gene expression was analyzed using RNA-Seq. For this purpose, BM-chimeric mice were used, whose bone marrow cells were replaced with green fluorescent protein (GFP)-expressing bone marrow cells to enable accurate and pure cell separation of microglia and mo-MΦ (FIGS. 5N-Q). A high degree of chimerism was achieved by using two sequential irradiations, the first consisting of low dose (300 rad) total body γ-irradiation, which induces lymphopenia and leads to lymphocyte extravasation from the lymph nodes (without inducing trafficking of immune cells to the CNS), and a second high dose γ-irradiation (950 rad), performed using head shielding to prevent blood brain barrier breakdown. After sorting of activated resident microglia and infiltrating mo-MΦ at different time points following SCI, the genome-wide expression profile of the distinct cell populations was determined (FIG. 2G). The present inventors goal was to test whether the gene expression imprint of long TGF-β1 exposure on nave myeloid cells, such as BM-MΦ (FIG. 2A), was similar to the unique gene expression signature of adult microglia during recovery from SCI, as these cells are chronically exposed to the CNS microenvironment. Specifically, the present inventors tested whether genes that were highly expressed by microglia compared to mo-MΦ over the course of the response to SCI (FIG. 2H; blue; p-value<0.05) overlapped with genes that were elevated ex vivo in BM-MΦ following long exposure to TGF-β1 (FIG. 2H; red; 2 fold). In parallel, the present inventors determined whether genes that were highly expressed by mo-MΦ compared to microglia over the course of the response to SCI (FIG. 2I; blue; p-value<0.05) overlapped with genes that were reduced ex vivo by TGF-β1 in BM-MΦ that were not previously exposed to this cytokine (FIG. 2I; red; 2 fold). Global comparison, demonstrated a significant overlap among the up-regulated genes (p<10⁻⁵), as seen by the intersecting genes (FIG. 2H; Tables 2 and 3, below). Next, the present inventors focused on genes that were altered by TGF-β1 and might affect the microglial phenotype during the repair process (at days 3 and 7). 20 genes were identified that were decreased by TGF-β1 treatment (2 fold) ex vivo, and were expressed at low levels (2 fold) by microglia (FIG. 2J), and 41 genes that were elevated by TGF-β1 and were more highly expressed in microglia in vivo compared to mo-MΦ during days 3 and 7 following SCI (FIG. 2K); among them were important immune response mediators, and transcription factors (TFs). The comparison between the signature of TGF-β1 on nave myeloid cells and the CNS imprint on the resident microglia during the repair process suggested that TGF-β1 has a significant role in shaping the adult microglial response under pathology.

TABLE 2
Intersection between in vivo gene expression of the cells
and ex vivo up-regulated genes following TGF-β1 kinetic

| | | | | | |
|---|---|---|---|---|---|
| 5730528L13RIK | Csf1 | Gramd1A | Mlxipl | Pygl | Trp53inp2 |
| Abca7 | Cspg4 | H2afv | Mtus1 | Rcsd1 | Ulk1 |
| Abcd2 | Ctsf | Hdac4 | Nat81 | Rhob | Usp35 |
| Abtb1 | Cxcr4 | Hist1h1c | Ncf1 | Rock2 | Vsp37b |
| Agap1 | Dedd2 | Hist1h2bc | Ndc80 | Rrm2 | Wipi1 |
| Ankrd33b | Dennd2A | Hmgn5 | Nlrx1 | Satb1 | Ypel3 |
| Arap3 | Dmwd | Hp | Notch1 | Sept5 | Zzeb1 |
| Arid3a | Dock8 | Id1 | Osgin1 | Sepx1 | Zfp688 |
| Arid3b | Dok3 | Ier2 | Pacs1 | Sesn2 | Zfyve1 |
| Arsb | Dst | Igf1r | Paqr7 | Sh2b1 | Zmpste24 |
| Atg16l2 | Ehd1 | Il17ra | Parvg | Sh3bp5 | Zyx |
| Bc017647 | F630028O10RIK | Inpp1 | Pbk | Slc16a3 | |
| Bc037034 | Fam107b | Irf2bp1 | Pbx1 | Slc16a6 | |
| Brd3 | Fam125b | Itgb3 | Pbxip1 | Slc41a3 | |
| Btg2 | Fam160b2 | Itprip | Pde2a | Snx11 | |
| Ccdc34 | Fbxo31 | Junb | Pdlim7 | Sort1 | |
| Ccpg1 | Fkbp5 | Kif21b | Pfkfb4 | Stx11 | |
| Cd24a | Fut7 | Kif22 | Phf21a | Tcp11l2 | |
| Cd300lf | Gadd45a | Klf2 | Phospho1 | Tk1 | |
| Cdc20 | Gadd45g | Klf7 | Phyhd1 | Tle3 | |
| Cdk17 | Gfod1 | Lasp1 | Pknox1 | Tmem119 | |
| Cdk5rl | Glipr2 | Ldhb | Plk3 | Tmem63a | |
| Cebpd | Gp1bb | Lime1 | Pnpla7 | Tnf | |
| Cib2 | Gpr56 | Lmbr11 | Ppp1r15a | Tnfsf14 | |
| Coq4 | Gpr84 | Mgst1 | Ptgs1 | Tnk2 | |

Of note, Table 2 provides a list of 136 genes of the intersection between the genes that were up-regulated in BM-MΦ at least 2 fold due to the exposure to TGF-β1 ex vivo, and genes from the in vivo kinetic that their expression was significantly different (p-value<0.05) between microglia and mo-MΦ, and among them only those that were expressed to higher extent in microglia compared to mo-MΦ along the kinetic following SCI.

TABLE 3
Intersection between in vivo gene expression of the cells
and ex vivo down-regulated genes following TGF-β1 kinetic.

| | | | | |
|---|---|---|---|---|
| 1700030F18RIK | Dapp1 | Ifi27l2a | Phactr4 | Stx3 |
| 5031439G07RIK | Ddx27 | Ifitm3 | Pip4k2a | Tia1 |
| Acat1 | Dna2 | Itgb7 | Plekha1 | Tifa |
| Ankrd57 | Dse | Jarid2 | Plin2 | Tmem176a |
| Arhgap17 | Emp1 | Klf13 | Pml | Tmem176b |
| Atf3 | Eps8 | Lgals3 | Ppap2c | Tmem51 |
| Batf3 | Ero1Lb | Lipa | Ppdpf | Tmsb10 |
| Cars | Evi2a | Lrrc33 | Ptpn2 | Tob2 |
| Ccl9 | Evl | Maf | Ptpro | Tusc1 |
| Ccr5 | Fam46a | Mapk6 | Pvr | Uap1 |
| Cd300a | Flcn | Mbtd1 | Pyhin1 | Uvrag |
| Cd36 | Fnip2 | Metrnl | Rasgef1b | Wdr82 |
| Cd74 | Fyn | Mrpl36 | Rbpj | Yrdc |
| Cd93 | Gstp2 | Naa20 | Rgs1 | Zfand5 |
| Clec4a1 | Gvin1 | Necap1 | Rpl29 | Zfp524 |
| Clec4a3 | H1f0 | Nudt16 | Sat1 | |
| Csda | Hmga1 | Parp9 | Slfn8 | |
| Cyth3 | Hmga1-rs1 | Pdcd2l | Solh | |
| D4wsu53e | Idh1 | Pdxk | Spred1 | |

Of note, Table 3 provides list of 91 genes of the intersection between the genes that were down-regulated in BM-MΦ at least 2 fold due to the exposure to TGF-β1 ex vivo, and genes from the in vivo kinetic that their expression was significantly different (p-value<0.05) between microglia and mo-MΦ, and among them only those that were expressed to higher extent in mo-MΦ compared to microglia along the kinetic following SCI.

### Example 3

### The Transcription Factor IRF7 is Required for the M1-to-M2 Switch and is Suppressed by TGF-β1

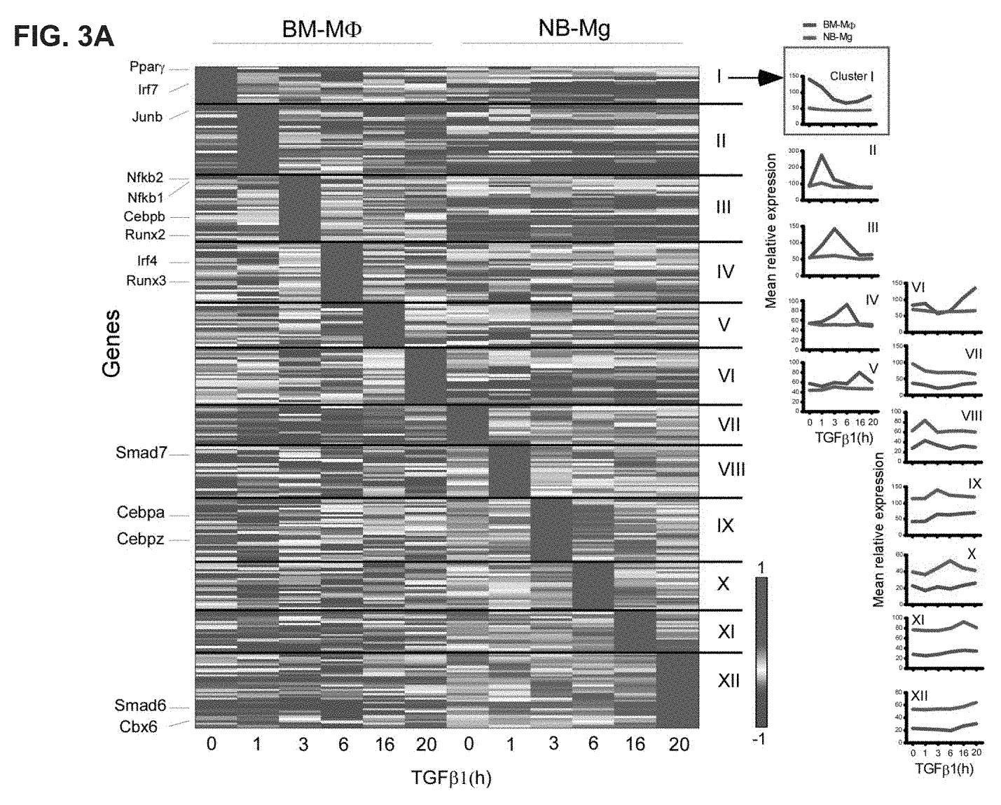

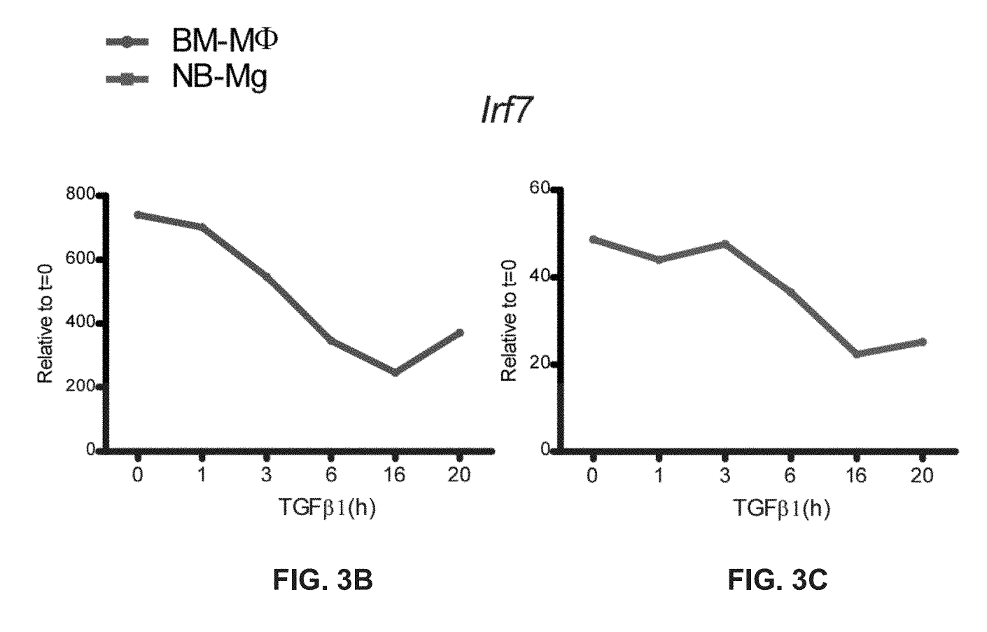

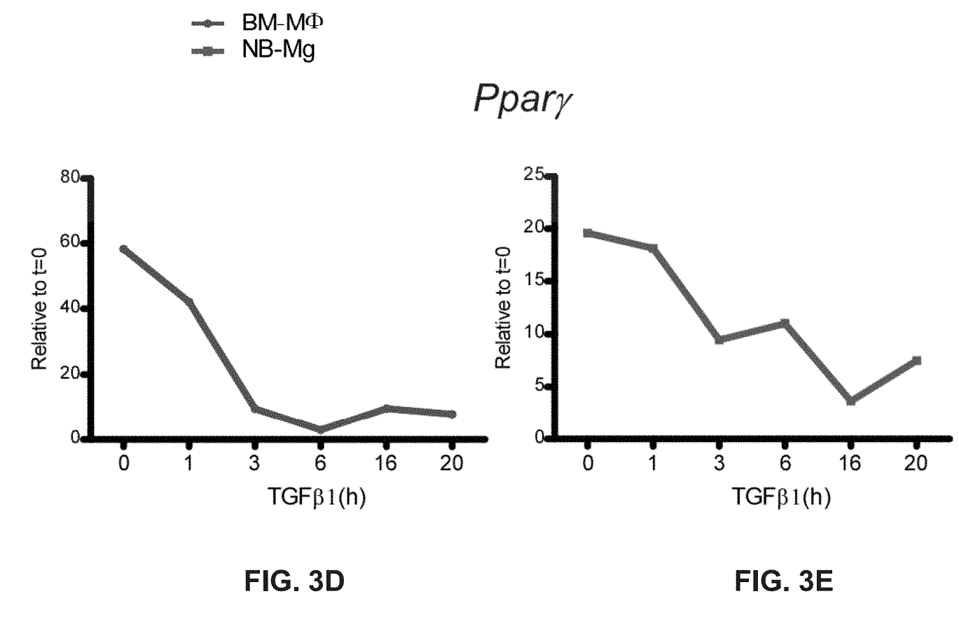

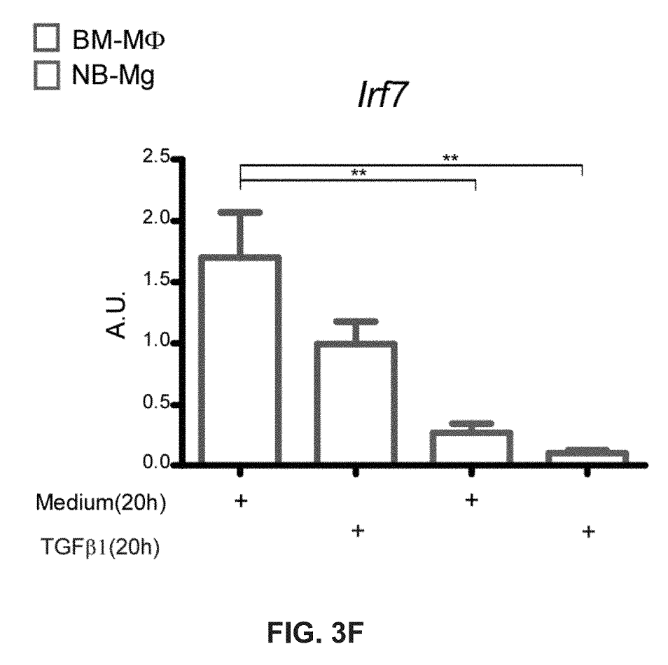

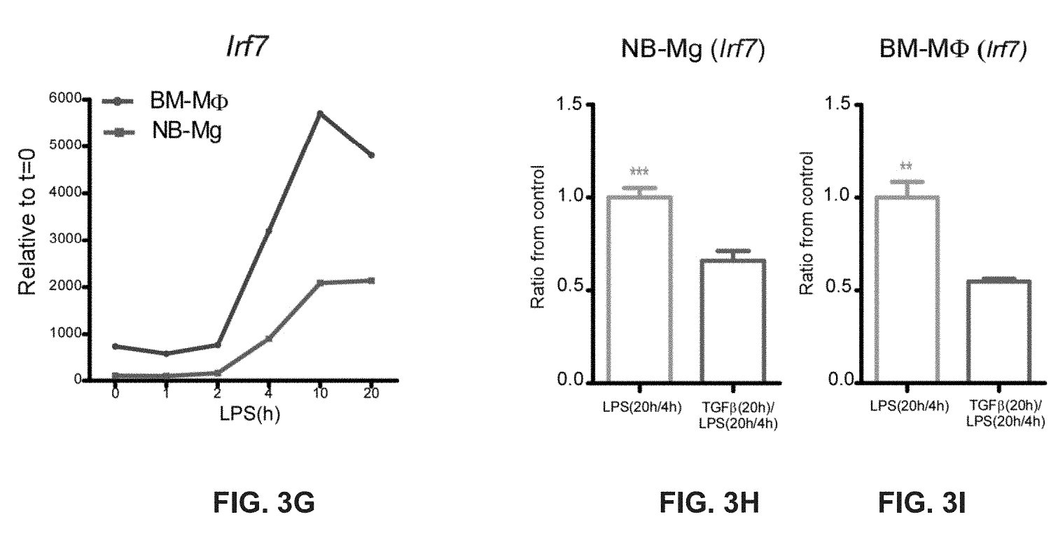

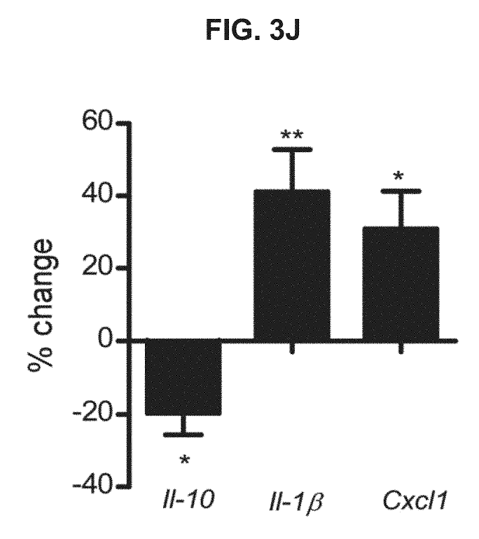

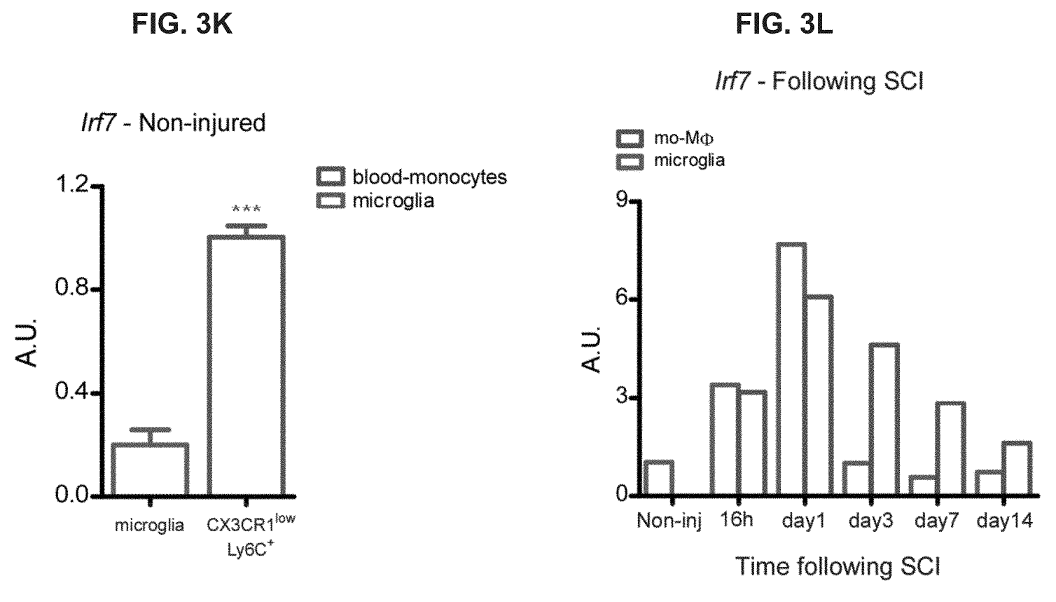

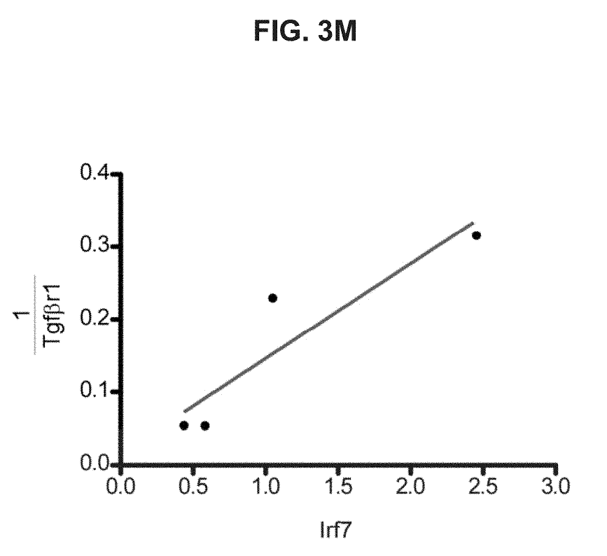

In order to understand the mechanism underlying TGF-β1 impairment of the M1-to-M2 switch, the global gene expression data was further analyzed, seeking TFs whose expression was altered by the extended exposure to TGF-β1, and that were also involved in the M2 polarization in the previous LPS paradigm (FIG. 3A). The present inventors first focused on clusters I and XII (FIG. 3A) in which the TFs were expressed by both naive BM-MΦ and NB-Mg, and were similarly and significantly changed along the time course of TGF-β1 treatment. Among these TFs, several candidates were identified in cluster I: peroxisome proliferator-activated receptor γ (Pparγ), a member of the nuclear receptor family of transcription factors that mediates transcriptional activation of anti-inflammatory genes, and Interferon regulatory factor 7 (Irf7), an essential TF for antiviral immunity, whose involvement in the regulation of the M1-to-M2 switch has not been previously reported. The expression of both TFs was down regulated ex vivo following TGF-β1 exposure (FIGS. 3B-F). Notably, analysis of the Irf7 expression profile in the ex vivo model following continuous exposure to LPS (FIG. 1A), revealed that Irf7 expression by both cell types was induced starting from the 4 hour time point, and remained high from 10 hours onward following long exposure to LPS, during the M2-polarization period. Yet, BM-MΦ expressed higher levels of Irf7 compared to NB-Mg along the entire time course (FIG. 3G). Importantly, however, under the same LPS paradigm, levels of Irf7 expression in both BM-MΦ and NB-Mg were reduced in cells preconditioned with TGF-β1 (FIGS. 3H-I). To substantiate the novel functional role of IRF7 in the M1-to-M2 conversion, small interfering RNA (siRNA) was used to silence Irf7 expression in BM-MΦ ex vivo (thereby reducing Irf7 levels in BM-MΦ to levels comparable to those in microglia), and tested its impact on the expression of pro- and anti-inflammatory genes (FIG. 3J). Importantly, Irf7 silencing decreased the levels of Il-10 expression and elevated the pro-inflammatory cytokine, 11-1β, and the chemokine Cxcl1, in the LPS tolerance model (FIG. 3J).

To further substantiate the present findings, attributing an important role to IRF7 in controlling microglial behavior at adulthood under injurious conditions, the present inventors first compared its expression levels by “resting” microglia isolated from adult spinal cord parenchyma of CX3CR1^{GFP/+} mice relative to nave circulating blood monocytes (CX3CR1^{low}Ly6C⁺). A higher level (approximately 5 fold) of Irf7 was observed in nave monocytes as compared to healthy adult spinal cord-derived microglia (FIG. 3K). Quantitative PCR analyses of isolated microglia and infiltrating mo-MΦ following SCI (FIG. 2A), revealed an increase in Irf7 expression on day 1, in both resident microglia and in the isolated mo-MΦ, which was rapidly reduced in microglia to basal levels, unlike in mo-MΦ, which maintained high levels of Irf7 expression from day 3 onward (p-value=0.051) (FIG. 3L). Interestingly, microglial behavior over the course of response to the injury revealed an inverse correlation between expression levels of Irf7 and that of the receptor to TGF-β1, Tgfβr1 (FIG. 3M), in line with the ex vivo observations (FIGS. 3B-F). Overall, these results suggest that Irf7, which was down regulated by TGF-β1 and whose expression was less pronounced in microglia during homeostasis and following SCI relative to mo-MΦ, might be a potential candidate for curtailing the M1-to-M2 circuit in myeloid cells. Therefore, Irf7 might be one of the factors that are modified in microglia by the CNS microenvironment, resulting in their inability to express the resolving phenotype under pathological conditions.

### Example 4

### IRF7 Regulates the M1-to-M2 Phenotype Switch Via Down-Regulation of the Pro Inflammatory Genes

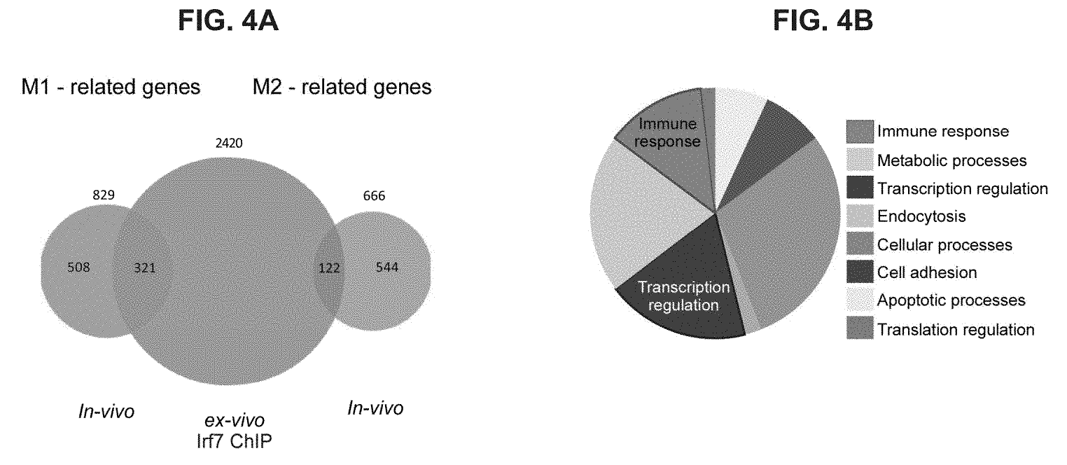

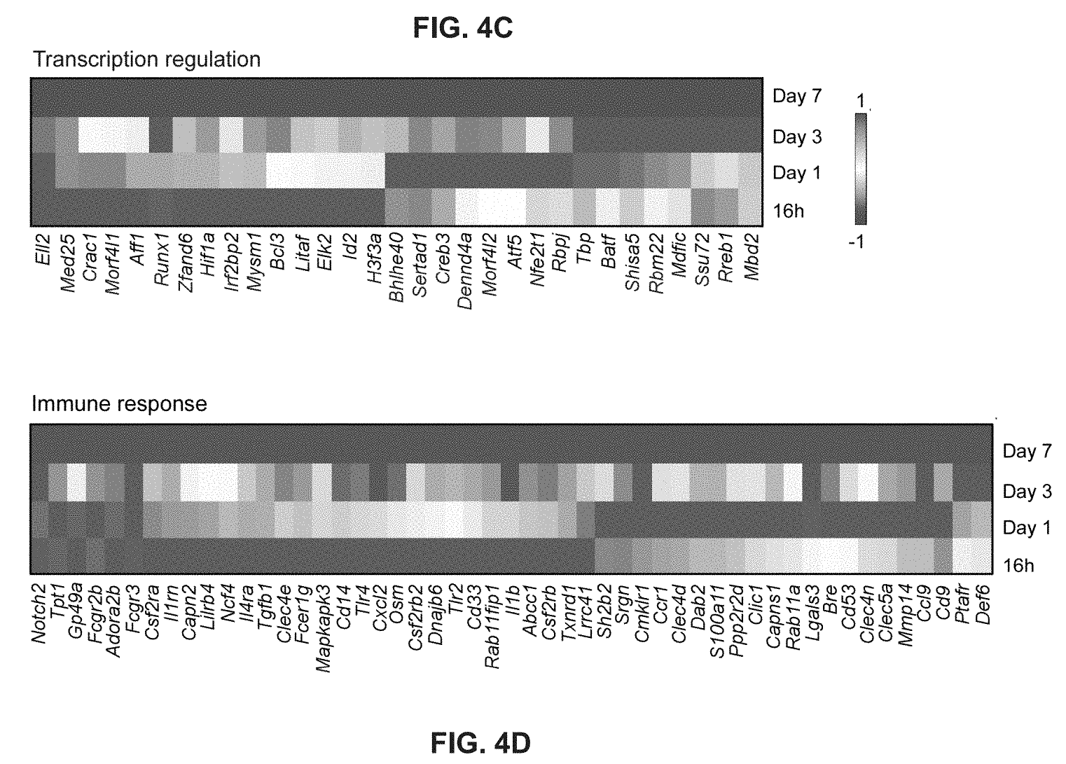

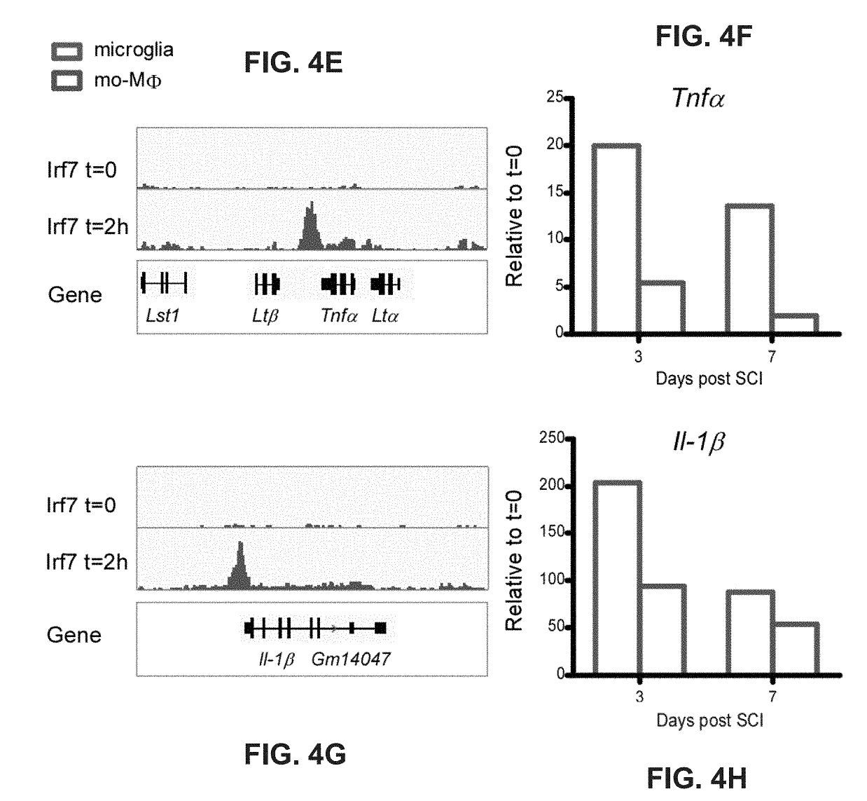

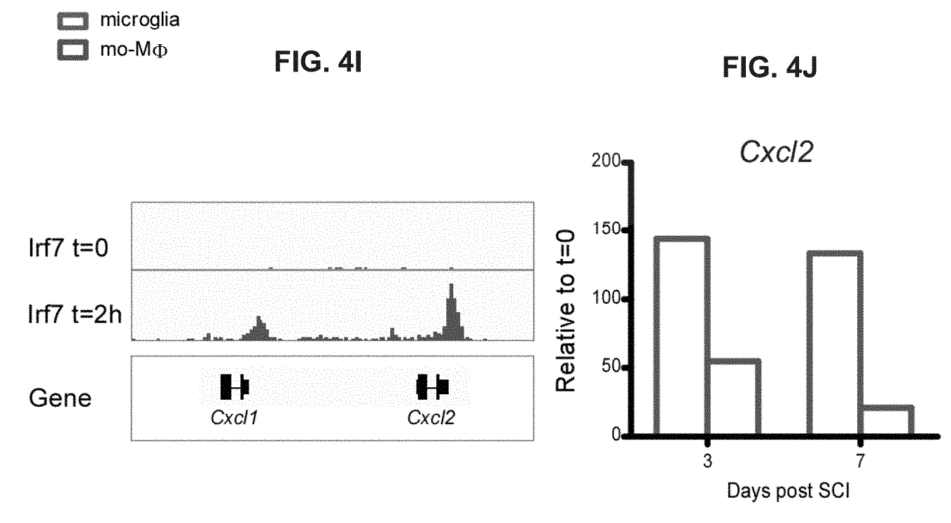

To identify the genes that are potentially directly regulated by Irf7, the present inventors next performed chromatin immunoprecipitation followed by massively parallel sequencing (ChIP-Seq) of LPS-treated (2 hour) myeloid cells. Since over the course of the recovery process following SCI, the mo-MΦ could differentiate to M1-like phenotype, at the early stage (days 1-3 post-injury), and M2-like phenotype, at the later stage (day 7 post-injury), the present inventors searched for the intersection of genes expressed at these days by the mo-MΦ in vivo with the Irf7 ChIP-Seq data. M1-related genes (FIG. 4A, green) were highly expressed at the initial days following the insult, while genes that were highly expressed at day 7 were classified as M2-related genes (FIG. 4A, orange). A significant intersection was found between the genes involved in the M1-response (that were down-regulated during the repair process), and genes whose promoters were found to bind Irf7 following LPS activation (FIG. 4A; p<10⁻⁵; and Tables 4A-C below). Those M1-related genes whose promoters were bound by Irf7 following LPS treatment were classified into functional groups using PANTHER gene ontology (FIG. 4B); among them, the present inventors focused on genes related to immune response and transcription regulation (FIGS. 4C-D and Tables 4A-C below). For example, the expression level of pro-inflammatory genes whose promoters were bound by Irf7, such as Cxcl1, Cxcl2, Il-1β and Tnfα, was higher in microglia compared to mo-MΦ at days 3 and 7 following SCI, substantiating the functional link between the lower expression level of Irf7 (FIG. 3L) and the pro-inflammatory profile observed in microglia at day 3 onwards following SCI (FIGS. 4E-J).

TABLE 4A
Intersection between in vivo gene expression of
mo-MΦ and ex vivo Irf7-ChIP assay.

| | | | | | Cellular adhesion |
|---|---|---|---|---|---|
| Metabolic processes | | | Transcription regulation | | and communication |
| Adprh | Gsr | Rragc | Aff1 | Morf412 | Rab21 |
| Alas1 | Hadhb | Sav1 | Atf5 | Msl1 | Rai14 |
| Aldoa | Hdlbp | Sgms2 | Batf | Mysm1 | Pef1 |
| Anxa2 | Imp4 | Slc3a2 | BC006779 | Nfe2l1 | Slc2a1 |
| Anxa7 | Isca1 | Sod2 | Bcl3 | Rhm22 | Alcam |
| Arf1 | Ldha | St3gal1 | Bhlhe40 | Rbpj | Arf4 |
| Asna1 | Lrp10 | Tpi1 | Cbfb | Rreb1 | Arl8a |
| Atp1a1 | Lsm14a | Txndc17 | Chrac1 | Runx1 | Cdk6 |
| Atp5b | Maea | Uba52 | Creb3 | Sertad1 | Dync1li1 |
| Atp5f1 | Mrpl39 | Vps54 | Dennd4a | Shisa5 | Fndc3a |
| Atp6v0c | Myo1c | Zc3hav1 | Elk3 | Sra1 | Fndc3b |
| Atp6v0e | Osbpl9 | Zfand3 | Ell2 | Ssu72 | Gna13 |
| Atp6v1g1 | Pcsk7 | Zfp207 | H3f3a | Tbp | Gnb1 |
| Cd38 | Pgam1 | | Hif1a | Zfand6 | Gng5 |
| Chst11 | Pgk1 | | Id2 | | Itgam |
| Cmpk1 | Pisd | | Irf2bp2 | | Itgb2 |
| Dtnbp1 | Psmb2 | | Litaf | | Lrrc8d |
| Esd | Ptpn2 | | Mbd2 | | Pip5k1c |
| Furin | Rabgef1 | | Mdfic | | Pvr |
| Gch1 | Rhbdf2 | | Med25 | | Sh3pxd2b |
| Glyr1 | Riok3 | | Med8 | | Ywhag |
| Gpd2 | Rpl7 | | Morf4l1 | | Zfp36l1 |

TABLE 4B
Intersection between in vivo gene expression of
mo-MΦ and ex vivo Irf7-ChIP assay.

| | | | Apoptotic | Translation | |
|---|---|---|---|---|---|
| Immune processes | | | processes | regulation | Endocytosis |
| Abcc1 | Def6 | Sh2b2 | Adam8 | Ddx17 | Actb |
| Adora2b | Dnajb6 | Srgn | Adam9 | Ddx5 | Actg1 |
| Bre | Fcer1g | Tgfb1 | Bag3 | Ddx58 | Actr10 |
| Capn2 | Fcgr2b | Tlr2 | Bax | Eif4a1 | Inpp5b |
| Capns1 | Fcgr3 | Tlr4 | Csrnp1 | Eif4a2 | Rhoa |
| Ccl9 | Gp49a | Tpt1 | Dad1 | | Snx18 |
| Ccr1 | Il1b | Txnrd1 | Dusp1 | | |
| Cd14 | Il1rn | | Dusp11 | | |
| Cd33 | Il4ra | | Jak2 | | |
| Cd53 | Lgals3 | | Lyn | | |
| Cd9 | Lilrb4 | | Malt1 | | |
| Clec4d | Lrrc41 | | Mcl1 | | |
| Clec4e | Mapkapk3 | | Pcbp1 | | |
| Clec4n | Mmp14 | | Pdcd6ip | | |
| Clec5a | Ncf4 | | Rnf34 | | |
| Clic1 | Notch2 | | Tmem49 | | |
| Cmklr1 | Osm | | Ube2a | | |
| Csf2ra | Ppp2r2d | | Ube2d2 | | |
| Csf2rb | Ptafr | | Ube2d3 | | |
| Csf2rb2 | Rab11a | | | | |
| Cxcl2 | Rab11fip1 | | | | |
| Dab2 | S100a11 | | | | |

TABLE 4C
Intersection between in vivo gene expression of
mo-MΦ and ex vivo Irf7-ChIP assay.

| Cellular processes | | | | Others | |
|---|---|---|---|---|---|
| Ap4b1 | Hnrnpf | Sumo1 | Mbd6 | 40787 | Grinl1a |
| Arcn1 | Hnrnpm | Tagln2 | Ostf1 | 1700020O03Rik | Gtpbp6 |
| Arpc1a | Ninj1 | Tes | Pfn1 | 1700123O20Rik | Mocs1 |
| Arpc2 | Nub1 | Tmod3 | Pnrc1 | 1810058I24Rik | Phf23 |
| Arpc3 | Ogt | Tomm20 | Psenen | 2010002N04Rik | Phlda1 |
| Cd274 | Pde4b | Tpm4 | Ptpn23 | 2010109K11Rik | Pid1 |
| Cfl1 | Pfdn2 | Tubb6 | Rab12 | 2310014H01Rik | Plekho2 |
| Chmp2b | Phrf1 | Ube2i | Rab20 | 2310016C08Rik | Rnf115 |
| Cltc | Ppp2cb | Vim | Rab32 | 2410004B18Rik | Rnf149 |
| Csnk2b | Psen1 | Btg1 | Rab7 | 2900073G15Rik | Sfrs3 |
| Cstb | Psmd8 | Tob2 | Snrpa | 4930471M23Rik | Stxbp3a |
| Cwc15 | Ptges3 | Atg9a | Spcs1 | 4933434E20Rik | Tacc1 |
| Dusp5 | Ptpmt1 | Fam129b | Stk24 | 6330578E17Rik | Tmem39a |
| Dync1i2 | Rnf4 | Fam63a | Syf2 | AW112010 | Tmem93 |
| Emp3 | Rnf5 | Fam96b | Tank | B230312A22Rik | Tmem9b |
| Erp44 | Rpl29 | Fem1c | | Ccdc109b | Tpt1p |
| Ezr | Slc15a3 | Glrx | | Ccdc115 | Txnip |
| Flot1 | Slc31a2 | Gm5506 | | Cdc42ep2 | Tyrobp |
| Fth1 | Slc35a4 | Gmeb2 | | Chic2 | Ufsp2 |
| Fxyd5 | Slc39a14 | Lasp1 | | Exoc8 | Wdr13 |
| G3bp1 | Slc7a11 | Lrrc59 | | Gm11428 | Yif1b |
| Hadha | Snx6 | Map2k1 | | Zc3h11a | |

Of note, Tables 4A-C provide a list of 321 genes of the intersection between the genes whose expression in vivo by mo-MΦ was decreased (M1-related genes) at day 7 relative to the first 3 days following SCI (p-value<0.05), and genes whose promoters were bound by the TF IRF7.

To elucidate a potential direct functional link between Irf7 expression levels and the ability of the microglia to switch from M1 to M2 phenotype, the present inventors examined whether induction of Irf7 using its well-known inducer, IFN-β1, would restore the ability of microglia to acquire an M2 phenotype ex vivo (e.g. elevating the levels of Irf7 in microglia to approach those found in macrophages) (FIG. 5A). The inventors first tested whether NB-Mg, which were exposed to TGF-β1, would be able to re-express Irf7 under the experimental paradigm. To this end, the cells were exposed to IFN-β1, a known inducer of Irf7. The results illustrate that tolerant NB-Mg preconditioned with TGF-β1 were able to express Irf7 following IFN-β1 stimulation (FIG. 5B). Further, it was observed that NB-Mg that were preconditioned with TGF-β1 and stimulated with IFN-β1 underwent M1-to-M2 phenotype switch. These microglia showed high expression levels of Il-10, and low expression levels of Cxcl1, Cxcl2 and Il-1β, compared to NB-Mg that were only preconditioned with TGF-β1 (FIGS. 5C-F). These results indicated that the effect of TGF-β1 on microglia could be abrogated by Irf7 induction, overcoming the inability to undergo an M1-to-M2 phenotype switch.

Finally, the inventors tested whether Irf7 induction in vivo would enable overcoming the microglia impairment to switch phenotype by down-regulating the expression levels of pro-inflammatory cytokines following SCI. To this end, spinally injured GFP>WT chimeric mice were locally injected with IFN-β1 (control mice were injected with PBS). Injections were performed directly into the parenchyma in order to elevate Irf7 expression in the inflammatory microglial cells located in close proximity to the lesion site. The time point for injection was determined based on the gene expression kinetics of inflammatory cytokines observed in microglia following SCI, in which it was found that Tnfα and Il-1β expression by sorted microglia peaked at day 1 and spontaneously, though not completely, resolved at day 3 following SCI (FIGS. 5G-I); therefore, IFN-β1 was injected 24 hours after the insult in order to increase Irf7 regulatory activity during the peak of microglial inflammation. Activated microglia were sorted (FIGS. 5N-Q) 48 hours and 72 hours following SCI from the lesion site area of the injured spinal cords, for RNA extraction and evaluation of Tnfα and Il-1β expression by RT-qPCR (FIG. 5G-I). Indeed, 12 hours following IFN-β1 injection (36 hours following SCI), sorted microglia from the lesion site area exhibited significantly elevated Irf7 expression levels compared to control PBS-injected mice (FIG. 5R). This induction of Irf7 expression by activated microglia was followed by significant reduction in Tnfα and Il-1β expression levels observed at 48 hours and 72 hours following SCI, compared to microglial cells derived from the PBS-injected mice (FIGS. 5J-M).

Overall, the present data demonstrated that the in vivo gene expression profile of adult resident microglia following CNS insult overlaps with the expression signature of myeloid cells that were exposed to TGF-β1. Moreover, it was shown that Irf7 plays a critical role in M1-to-M2 conversion of myeloid cells by negatively regulating expression of inflammatory pathway genes, such as Il-1β, Tnfα, Cxcl1 and Cxcl2, and up-regulating expression of anti-inflammatory genes, such as Il-10. Finally, the present results demonstrate that restoring Irf7 expression by IFN-β1 reactivates the circuits leading to M2 conversion by improving the resolution of pro-inflammatory cytokines expressed by microglia ex vivo and in vivo, following acute CNS insult.

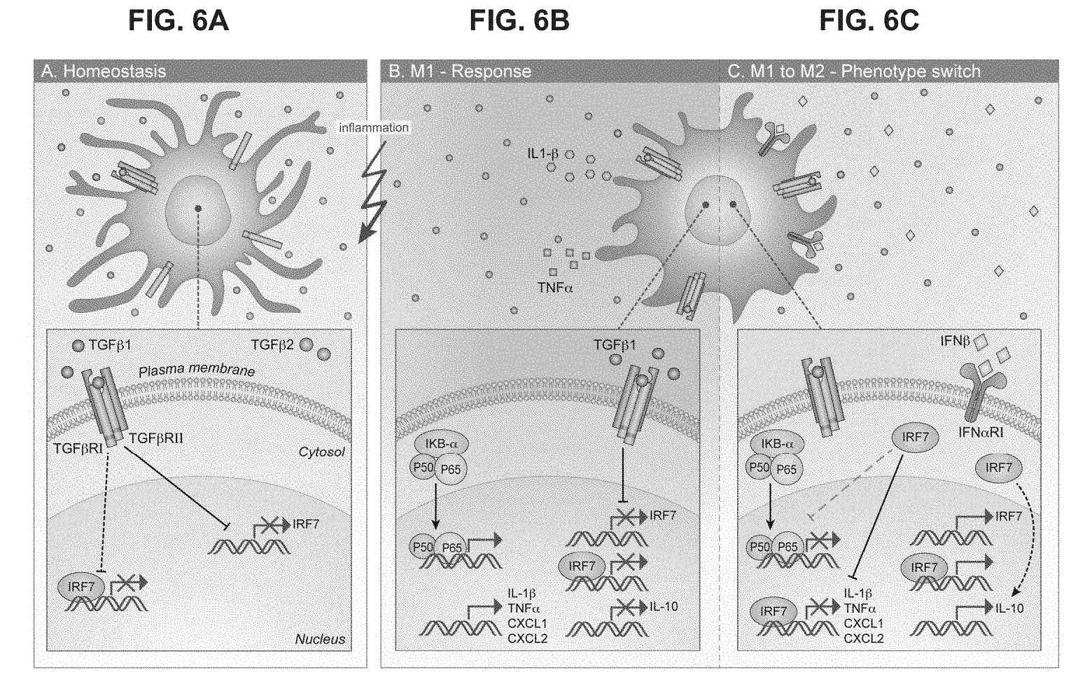

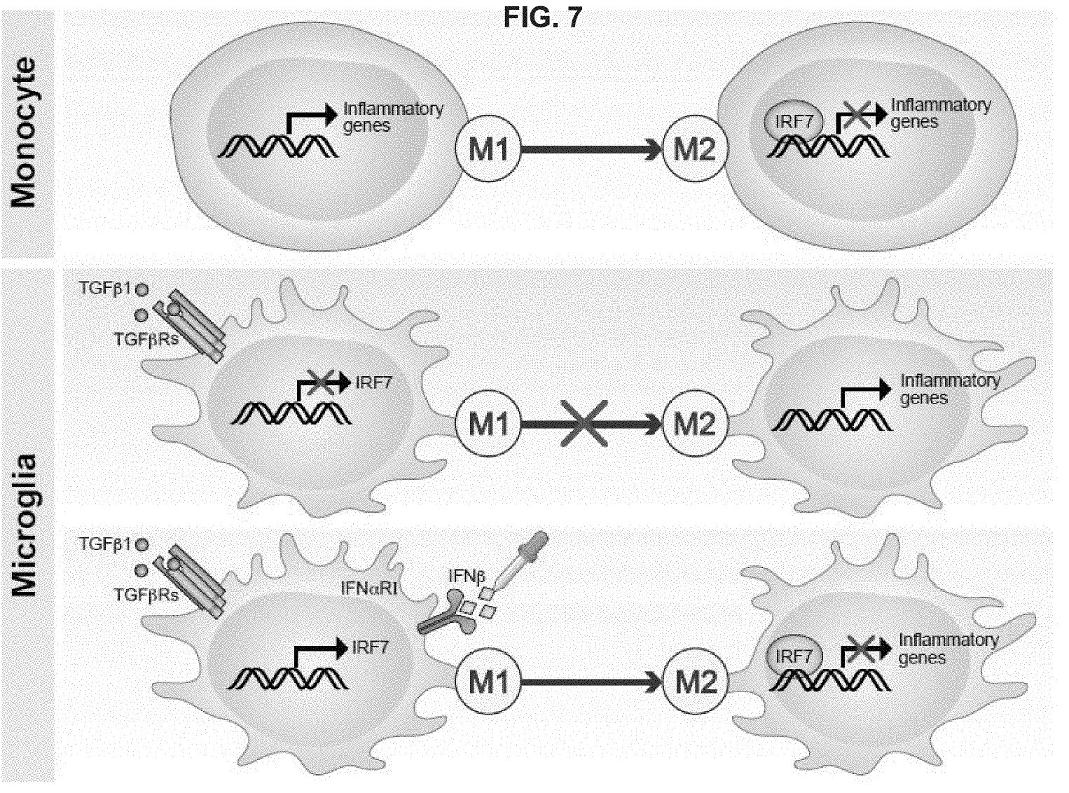

Taken together, the present study identifies a novel phenomenon of TGFβ1-induced tolerance, demonstrating that long exposure to TGF-β1 induces an altered state of responsiveness to anti-inflammatory signals. The present data revealed that beyond expression of distinctive markers during homeostasis, the TGF-β1-enriched environment impaired microglial ability to switch from M1-to-M2 phenotype under inflammatory conditions, through a reduction in Irf7 expression levels. These findings suggest that the circuitry underlying the exposure of microglia to TGF-β1 within the adult CNS microenvironment might be a double-edged sword, enabling their essential functions under normal physiological conditions, but imprinting incompetence to resolve inflammation under severe pathology. Thus, the tissue microenvironment may have a major effect on the phenotype of myeloid cells residing in it, not only during homeostasis, but also in their subsequent functional response to pathology. Interventions to alter these environmental effects, such as Irf7 induction in resident microglia, might have a therapeutic benefit in reducing CNS inflammation during pathology (FIGS. 6A-C and 7).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of upregulating an anti-inflammatory response in a central nervous system (CNS) of a subject in need thereof, the method comprising locally administering to the CNS of the subject a therapeutically effective amount of IFN-β, thereby upregulating the anti-inflammatory response in the CNS of the subject.

2. A method of treating an inflammation in a CNS of a subject in need thereof, the method comprising locally administering to the CNS of the subject a therapeutically effective amount of IFN-β, thereby treating the inflammation in the CNS of the subject.

3. A method of treating a disease, disorder, condition or injury of a CNS in a subject in need thereof, the method comprising locally administering to the CNS of the subject a therapeutically effective amount of IFN-β, thereby treating the disease, disorder, condition or injury of the CNS in the subject.

4-5. (canceled)

6. The method of claim 1, wherein said therapeutically effective amount upregulates the activity or expression of IRF7.

7. The method of claim 1, wherein said therapeutically effective amount downregulates the expression of at least one pro-inflammatory associated gene.

8. The method of claim 7, wherein said pro-inflammatory associated gene is selected from the group consisting of iNos, Tnfα, Il-1β, Il-6, Cxcl1, Cxcl2 and Cxcl10.

9. The method of claim 1, wherein said therapeutically effective amount upregulates the expression of at least one anti-inflammatory associated gene.

10. The method of claim 9, wherein said anti-inflammatory associated gene is selected from the group consisting of IL-10, MMR (CD206), CD36, DECTIN-1, IL-4 and IL-13.

11. The method of claim 1, wherein said therapeutically effective amount induces a M1-to-M2 phenotype conversion of a myeloid cell.

12. The method of claim 11, wherein said myeloid cell comprise a microglia cell.

13. The method claim 1, wherein said locally administering is to a parenchymal tissue of said CNS.

14. The method claim 1, wherein said locally administering is effected by a route selected from the group consisting of intracranial (IC), intracerebroventricular (ICV), intrathecal and intraparenchymal CSF administration.

15. The method of claim 1, wherein the subject is a human subject.

16. The method of claim 1, wherein the subject has a neurodegenerative disorder or a neuroinflammatory disorder.

17. The method of claim 1, wherein the subject has a disease, disorder, condition or injury of a CNS.

18. The method of claim 3, wherein said disease, disorder, condition or injury of said CNS is selected from the group consisting of spinal cord injury, closed head injury, blunt trauma, penetrating trauma, hemorrhagic stroke, ischemic stroke, cerebral ischemia, spinal ischemia, optic nerve injury, myocardial infarction.

19. The method of claim 1, wherein said IFN-β is soluble.
